(19) United States
(12) Patent Application Publication
(10) Pub. No.: US 2021/0023012 A1
(43) Pub. Date: Jan. 28, 2021

(54) Solid Pharmaceutical Compositions for Treating HCV

(71) Applicant: AbbVie Inc., North Chicago, IL (US)

(72) Inventors: Wei Liu, Riverwoods, IL (US); Hoi Kei Lon, North Chicago, IL (US); Sven Mensing, Ilvesheim (DE); Jeffrey Schmidt, Mount Prospect, IL (US); Neha Thakre, North Chicago, IL (US); Thin Yu Tu, Libertyville, IL (US)

(73) Assignee: AbbVie Inc., North Chicago, IL (US)

(21) Appl. No.: 16/837,576

(22) Filed: Apr. 1, 2020

Related U.S. Application Data
(60) Provisional application No. 62/830,926, filed on Apr. 8, 2019.

(51) Int. Cl. A61K 9/24; A61P 31/14; A61K 38/06; A61K 31/454; A61K 9/28; A61K 9/20; A61K 9/50; A61K 9/48

(52) U.S. Cl. CPC A61K 9/209; A61P 31/14; A61K 38/06; A61K 31/454; A61K 9/2866; A61K 9/4858; A61K 9/2013; A61K 9/2027; A61K 9/5047; A61K 9/485; A61K 9/4866; A61K 9/2009

(57) ABSTRACT

The present invention features solid pharmaceutical compositions comprising Compound 1 and Compound 2. In one embodiment, the solid pharmaceutical composition includes (1) a first type of film-coated granules which comprise 50 mg of Compound 1, as well as a pharmaceutically acceptable hydrophilic polymer and a pharmaceutically acceptable surfactant, all of which are formulated in amorphous solid dispersion; and (2) a second type of film-coated granules which comprise 20 mg of Compound 2, as well as a pharmaceutically acceptable hydrophilic polymer and a pharmaceutically acceptable surfactant, all of which are formulated in amorphous solid dispersion.

### FIELD OF THE INVENTION

The present invention relates to solid pharmaceutical compositions comprising anti-HCV compounds and methods of using the same for treating HCV infection.

### BACKGROUND OF THE INVENTION

The hepatitis C virus (HCV) is an RNA virus belonging to the Hepacivirus genus in the Flaviviridae family. The enveloped HCV virion contains a positive stranded RNA genome encoding all known virus-specific proteins in a single, uninterrupted, open reading frame. The open reading frame comprises approximately 9500 nucleotides and encodes a single large polyprotein of about 3000 amino acids. The polyprotein comprises a core protein, envelope proteins E1 and E2, a membrane bound protein p7, and the non-structural proteins NS2, NS3, NS4A, NS4B, NS5A and NS5B.

Chronic HCV infection is associated with progressive liver pathology, including cirrhosis and hepatocellular carcinoma. Chronic hepatitis C may be treated with peginterferon-alpha in combination with ribavirin. Substantial limitations to efficacy and tolerability remain as many users suffer from side effects, and viral elimination from the body is often incomplete. Also, although there are commercially available therapies for adults and pediatric populations aged 12-18, few options are available for pediatric populations aged 3-11. Therefore, there is a need for new drugs to treat HCV infection for these pediatric subpopulations.

### SUMMARY OF THE INVENTION

The present invention features solid pharmaceutical compositions comprising Compound 1 and Compound 2. In one embodiment, the solid pharmaceutical composition includes (1) a first type of film-coated granules which comprise 50 mg of Compound 1, as well as a pharmaceutically acceptable hydrophilic polymer and a pharmaceutically acceptable surfactant, all of which are formulated in amorphous solid dispersion; and (2) a second type of film-coated granules which comprise 20 mg of Compound 2, as well as a pharmaceutically acceptable hydrophilic polymer and a pharmaceutically acceptable surfactant, all of which are formulated in amorphous solid dispersion.

In one embodiment, the present invention provides a method for treating hepatitis C virus (HCV) infection in a pediatric patient, comprising administering (1) Compound 1 and (2) Compound 2, wherein (i) the patient is from 3 years old to less than 6 years old, Compound 1 is administered at a dose of about 150 mg, and Compound 2 is administered at a dose of about 60 mg; (ii) the patient is from 6 years old to less than 9 years old, Compound 1 is administered at a dose of about 200 mg, and Compound 2 is administered at a dose of about 80 mg; or (iii) the patient is from 9 years old to less than 12 years old, Compound 1 is administered at a dose of about 250 mg, and Compound 2 is administered at a dose of about 100 mg.

In one embodiment, the present invention provides a method for treating hepatitis C virus (HCV) infection in a pediatric patient, comprising administering a film-coated granule composition comprising
50 mg of Compound 1 and 20 mg of Compound 2, wherein the film-coated granule composition is provided in a sachet, and wherein the patient is from 3 years old to less than 6 years old and three sachets are administered, comprising a total of about 150 mg of Compound 1, and about 60 mg of Compound 2, and the patient obtains a sustained virologic response about 12 weeks post treatment (SVR12).

In one embodiment, the present invention provides a method for treating hepatitis C virus (HCV) infection in a pediatric patient, comprising administering a film-coated granule composition comprising
50 mg of Compound 1 and 20 mg of Compound 2, wherein the film-coated granule composition is provided in a sachet, and wherein the patient is from 6 years old to less than 9 years old and four sachets are administered, comprising a total of about 200 mg of Compound 1, and about 80 mg of Compound 2, and the patient obtains a sustained virologic response about 12 weeks post treatment (SVR12).

In one embodiment, the present invention provides a method for treating hepatitis C virus (HCV) infection in a pediatric patient, comprising administering a film-coated granule composition comprising 50 mg of Compound 1 and 20 mg of Compound 2, wherein the film-coated granule composition is provided in a sachet, and wherein the patient is from 9 years old to less than 12 years old and five sachets are administered, comprising a total of about 250 mg of Compound 1, and about 100 mg of Compound 2, and the patient obtains a sustained virologic response about 12 weeks post treatment (SVR12).

In another embodiment, the patient is from 3 years old to less than 6 years old, Compound 1 is administered at a dose of about 150 mg, and Compound 2 is administered at a dose of about 60 mg.

In yet another embodiment, the patient is from 6 years old to less than 9 years old, Compound 1 is administered at a dose of about 200 mg, and Compound 2 is administered at a dose of about 80 mg.

In another embodiment, the patient is from 9 years old to less than 12 years old, Compound 1 is administered at a dose of about 250 mg, and Compound 2 is administered at a dose of about 100 mg.

In one embodiment, Compound 1 is administered from a first type of film-coated granule comprising an amorphous solid dispersion including (i) Compound 1, (ii) copovidone and (iii) Vitamin E TPGS. Further, the total amount of Compound 1 comprised in the first type of granules is 50 mg.

In another embodiment, Compound 2 is administered from a second type of film-coated granules comprising an amorphous solid dispersion including (i) Compound 2, (ii) copovidone and (iii) Vitamin E TPGS and propylene glycol monocaprylate. Further, the total amount of Compound 2 comprised in the second type of granules is 20 mg.

One embodiment, provides a solid pharmaceutical composition comprising: (1) 50 mg of Compound 1 formulated in amorphous solid dispersion which further comprises from 50% to 80% by weight of a first pharmaceutically acceptable polymer and from 5% to 15% by weight of a first pharmaceutically acceptable surfactant; and (2) 20 mg of Compound 2 formulated in amorphous solid dispersion which further comprises from 50% to 90% by weight of a second pharmaceutically acceptable polymer and from 5% to 15% by weight of a second pharmaceutically acceptable surfactant. Further, in one embodiment, the composition is a mixture of (1) a first type of film-coated granules including said 50 mg of Compound 1 and (2) a second type of film-coated granules including said 20 mg of Compound 2. Furthermore, in one embodiment, the amorphous solid dispersion in which Compound 1 is formulated comprises 20% by weight of Compound 1, and the amorphous solid dispersion in which Compound 2 is formulated comprises 10% by weight of Compound 2. In one embodiment, the composition is a mixture of (1) a first type of film-coated granules including said 50 mg of Compound 1 and (2) a second type of film-coated granules including said 20 mg of Compound 2. Further said first and second polymers are copovidone, and said first and second surfactants are Vitamin E TPGS. In one embodiment, said first and second polymers are copovidone, and said first surfactant is Vitamin E TPGS, and said second surfactant is a combination of Vitamin E TPGS and propylene glycol monocaprylate.

Another embodiment, provides a stable, oral, immediate release solid pharmaceutical composition comprising: (1) 50 mg of Compound 1 formulated in an amorphous solid dispersion which further comprises from 50% to 80% by weight of a first pharmaceutically acceptable polymer and from 5% to 15% by weight of a first pharmaceutically acceptable surfactant; and (2) 20 mg of Compound 2 formulated in an amorphous solid dispersion which further comprises from 50% to 90% by weight of a second pharmaceutically acceptable polymer and from 5% to 15% by weight of a second pharmaceutically acceptable surfactant, wherein the composition is provided in a sachet and is stable for the duration of a shelf life of about 24 months in the sachet.

In one embodiment, the composition is a mixture of (1) a first type of film-coated granule including the 50 mg of Compound 1 and (2) a second type of film-coated granule including the 20 mg of Compound 2.

In one embodiment, the amorphous solid dispersion in which Compound 1 is formulated comprises 20% by weight of Compound 1, and the amorphous solid dispersion in which Compound 2 is formulated comprises 10% by weight of Compound 2.

In one embodiment, the composition is a mixture of (1) a first type of film-coated granule including the 50 mg of Compound 1 and (2) a second type of film-coated granule including the 20 mg of Compound 2.

In one embodiment, the first and second polymers are copovidone, and the first and second surfactants are Vitamin E TPGS.

In one embodiment, the first and second polymers are copovidone, and the first surfactant is Vitamin E TPGS, and the second surfactant is a combination of Vitamin E TPGS and propylene glycol monocaprylate.

In one embodiment, the composition has an in vitro release profile according to at least one of the following profiles: (i) when the composition is dissolved in 1000 mL of a dissolution medium using a standard USP dissolution Apparatus 2 (paddle) with Japanese sinker operating at 75 RPM at 37° C., at least 80% of Compound 1 in the composition is released within 3 hours and at least 80% of Compound 2 in the composition is released within 3 hours, wherein the dissolution medium is 0.1 M Acetate buffer (pH 4.0) with 1% Polysorbate 80; (ii) when the composition is dissolved in 1000 mL of a dissolution medium using a standard USP dissolution Apparatus 2 (paddle) with Japanese sinker operating at 75 RPM at 37° C., at least 30% of Compound 1 in the composition is released within 50 minutes and at least 45% of Compound 2 in the composition is released within 50 minutes, wherein the dissolution medium is 0.1 M Acetate buffer (pH 4.0) with 1% Polysorbate 80; or (iii) when the composition is dissolved in 1000 mL of a dissolution medium using a standard USP dissolution Apparatus 2 (paddle) with Japanese sinker operating at 75 RPM at 37° C., at least 5% of Compound 1 in the composition is released within 25 minutes and at least 10% of Compound 2 in the composition is released within 25 minutes, wherein the dissolution medium is 0.1 M Acetate buffer (pH 4.0) with 1% Polysorbate 80.

In one embodiment, the composition has an in vitro release profile according to at least one of the following profiles: (i) when the composition is dissolved in 500 mL of a dissolution medium using a standard USP dissolution Apparatus 1 (basket) operating at 75 RPM at 37° C., at least 80% of Compound 1 in the composition is released within 40 minutes and at least 80% of Compound 2 in the composition is released within 40 minutes, wherein the dissolution medium is 0.1 M Acetate buffer (pH 4.0) with 1% Polysorbate 80; (ii) when the composition is dissolved in 500 mL of a dissolution medium using a standard USP dissolution Apparatus 1 (basket) operating at 75 RPM at 37° C., at least 30% of Compound 1 in the composition is released within 20 minutes and at least 45% of Compound 2 in the composition is released within 20 minutes, wherein the dissolution medium is 0.1 M Acetate buffer (pH 4.0) with 1% Polysorbate 80; or (iii) when the composition is dissolved in 500 mL of a dissolution medium using a standard USP dissolution Apparatus 1 (basket) operating at 75 RPM at 37° C., at least 5% of Compound 1 in the composition is released within 10 minutes and at least 10% of Compound 2 in the composition is released within 10 minutes, wherein the dissolution medium is 0.1 M Acetate buffer (pH 4.0) with 1% Polysorbate 80.

In one embodiment, a single dose of three sachets administered to a population of healthy, non-fasted patients from 3 years old to less than 6 years old results in a mean AUC value between about 6936 ng·h/mL and about 10838 ng·h/mL for Compound 1, and a mean AUC value between about 1840 ng·h/mL and about 2875 ng·h/mL for Compound 2.

In one embodiment, a single dose of four sachets administered to a population of healthy, non-fasted patients from 6 years old to less than 9 years old results in a mean AUC value between about 4776 ng·h/mL and about 7463 ng·h/mL for Compound 1, and a mean AUC value between about 1216 ng·h/mL and about 1900 ng·h/mL for Compound 2.

In one embodiment, a single dose of five sachets administered to a population of healthy, non-fasted patients from 9 years old to less than 12 years old results in a mean AUC value between about 5360 ng·h/mL and about 8375 ng·h/mL for Compound 1, and a mean AUC value between about 1328 ng·h/mL and about 2075 ng·h/mL for Compound 2.

Another embodiment provides a pharmaceutical composition that is bioequivalent to the composition described herein.

Another embodiment provides method for treating hepatitis C virus (HCV) infection, comprising administering the solid pharmaceutical composition described herein to a patient in need thereof, wherein the patient obtains a sustained virologic response about 12 weeks post treatment (SVR12).

Another embodiment provides a dispensing container containing a solid pharmaceutical composition described above. Further, the dispensing container is a sachet.

One embodiment provides a method for treating hepatitis C virus (HCV) infection, comprising administering a solid pharmaceutical composition as described above to a patient in need thereof.

Yet another embodiment provides a solid pharmaceutical composition comprising: (1) 50 mg of Compound 1 formulated in amorphous solid dispersion which further comprises from 50% to 80% by weight of a first pharmaceutically acceptable polymer and from 5% to 15% by weight of a first pharmaceutically acceptable surfactant; (2) 20 mg of Compound 2 formulated in amorphous solid dispersion which further comprises from 50% to 90% by weight of a second pharmaceutically acceptable polymer and from 5% to 15% by weight of a second pharmaceutically acceptable surfactant; and wherein the composition is provided in a dispensing container comprising a sachet. In another embodiment, the composition provides a mixture of (1) a first type of film-coated granules including said 50 mg of Compound 1 and (2) a second type of film-coated granules including said 20 mg of Compound 2.

The above objectives of the present invention is not intended to be exhaustive or to limit the invention to the precise one disclosed. Modifications and variations are possible in light of the teachings or may be acquired from practice of the invention. Thus, it is noted that the scope of the invention is defined by the claims and their equivalents.

### BRIEF DESCRIPTION OF THE DRAWINGS

FIG. 1 and FIG. 2 each depict an exemplary sachet for use as a dispensing container in accordance with the oral dosage forms (e.g., film-coated granules) described herein.

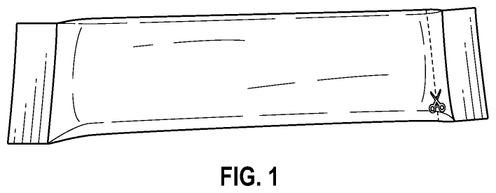

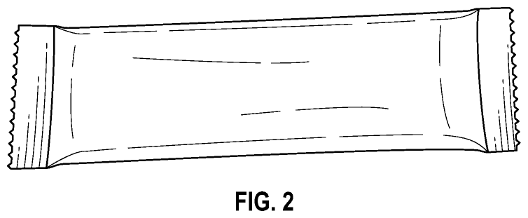

### DETAILED DESCRIPTION

The present invention features solid pharmaceutical compositions useful for treating HCV. These solid pharmaceutical compositions comprise:

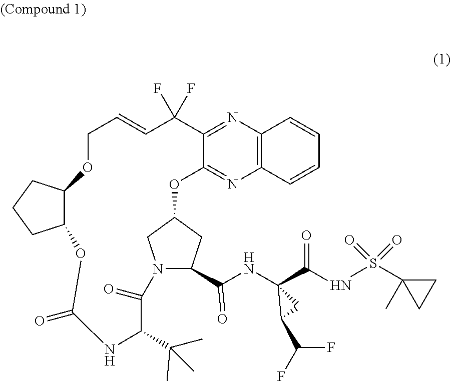 [SMILES: CC(C)(C)[C@@H]1NC(=O)O[C@@H]2CCC[C@H]2OC/C=C/C(F)(F)c2nc3ccccc3nc2O[C@@H]2C[C@@H](C(=O)N[C@]3(C(=O)NS(=O)(=O)C4(C)CC4)C[C@H]3C(F)F)N(C2)C1=O]

or a pharmaceutically acceptable salt thereof, formulated in amorphous solid dispersion, and

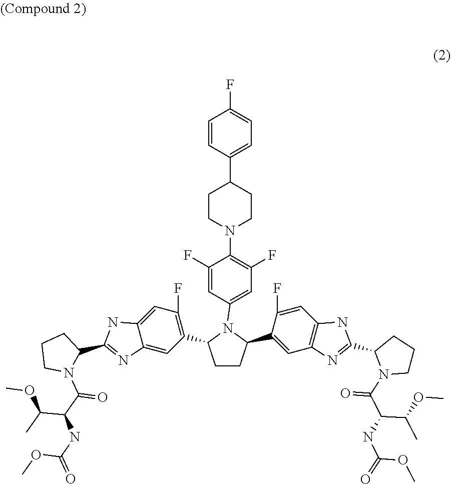

or a pharmaceutically acceptable salt thereof, formulated in amorphous solid dispersion.

Compound 1 is a potent HCV protease inhibitor and is described in U.S. Patent Application Publication No. 2012/0070416, which is incorporated herein by reference in its entirety. Compound 2 is a potent NS5A inhibitor and is described in U.S. Patent Application Publication No. 2012/0220562, which is incorporated herein by reference in its entirety. In one embodiment, the invention provides a stable, oral, immediate release solid pharmaceutical composition comprising:
(1) 50 mg of Compound 1 formulated in an amorphous solid dispersion which further comprises from 50% to 80% by weight of a first pharmaceutically acceptable polymer and from 5% to 15% by weight of a first pharmaceutically acceptable surfactant; and
(2) 20 mg of Compound 2 formulated in an amorphous solid dispersion which further comprises from 50% to 90% by weight of a second pharmaceutically acceptable polymer and from 5% to 15% by weight of a second pharmaceutically acceptable surfactant,
   wherein the composition is provided in a sachet and is stable for the duration of a shelf life of about 24 months in the sachet.

In one embodiment, Compound 1 and Compound 2 are separately formulated in different amorphous solid dispersions. These solid dispersions are then milled and/or mixed with other excipients, to form a solid pharmaceutical composition that contains both Compound 1 and Compound 2.

In another embodiment, Compound 1 and Compound 2 are separately formulated in different amorphous solid dispersions. The solid dispersion comprising Compound 1 is milled and/or mixed with other excipients, and then compressed into a first layer of a tablet; and the solid dispersion comprising Compound 2 is likewise milled and/or mixed with other excipients, and compressed into a second layer of the same tablet.

In another embodiment, Compound 1 and Compound 2 are separately formulated in different amorphous solid dispersions. The solid dispersion comprising Compound 1 is milled and/or mixed with other excipients, and then compressed into mini-tablets, and each mini-tablet is no more than 5 mm in size. The solid dispersion comprising Compound 2 is likewise milled and/or mixed with other excipients, and compressed into mini-tablets, and each mini-tablet is no more than 5 mm in size. The mini-tablets containing Compound 1 are then mixed with the mini-tablets containing Compound 2, to provide the desired dosing for Compound 1 and Compound 2.

In another embodiment, Compound 1 and Compound 2 are separately formulated in different amorphous solid dispersions. The solid dispersion comprising Compound 1 is milled and/or mixed with other excipients, and then compressed into mini-tablets, and each mini-tablet is no more than 3 mm in size. The solid dispersion comprising Compound 2 is likewise milled and/or mixed with other excipients, and compressed into mini-tablets, and each mini-tablet is no more than 3 mm in size. The mini-tablets containing Compound 1 are then mixed with the mini-tablets containing Compound 2, to provide the desired dosing for Compound 1 and Compound 2.

In another embodiment, Compound 1 and Compound 2 are separately formulated in different amorphous solid dispersions. The solid dispersion comprising Compound 1 is milled and/or mixed with other excipients, and then compressed into mini-tablets, and each mini-tablet is no more than 2 mm in size. The solid dispersion comprising Compound 2 is likewise milled and/or mixed with other excipients, and compressed into mini-tablets, and each mini-tablet is no more than 2 mm in size. The mini-tablets containing Compound 1 are then mixed with the mini-tablets containing Compound 2, to provide the desired dosing for Compound 1 and Compound 2.

In another embodiment, Compound 1 and Compound 2 are separately formulated in different amorphous solid dispersions. The solid dispersion comprising Compound 1 is milled and/or mixed with other excipients and then compressed to form granules. The granules containing Compound 1 are then coated with a non-functional film coating. The solid dispersion comprising Compound 2 is likewise milled and/or mixed with other excipients and then compressed form granules. The granules containing Compound 2 are then coated with a non-functional film coating. The film-coated granules containing Compound 1 are then mixed with the film-coated granules containing Compound 2 (e.g., in a dispensing container such as a sachet) to provide the desired dosing for Compound 1 and Compound 2.

Yet another embodiment provides a solid pharmaceutical composition comprising: (1) 50 mg of Compound 1 formulated in amorphous solid dispersion which further comprises from 50% to 80% by weight of a first pharmaceutically acceptable polymer and from 5% to 15% by weight of a first pharmaceutically acceptable surfactant; (2) 20 mg of Compound 2 formulated in amorphous solid dispersion which further comprises from 50% to 90% by weight of a second pharmaceutically acceptable polymer and from 5% to 15% by weight of a second pharmaceutically acceptable surfactant; and wherein the composition is provided in a dispensing container comprising a sachet. In another embodiment, the composition provides a mixture of (1) a first type of film-coated granules including said 50 mg of Compound 1 and (2) a second type of film-coated granules including said 20 mg of Compound 2.

In yet another embodiment, Compound 1 and Compound 2 are formulated in the same amorphous solid dispersion. The solid dispersion is milled and/or mixed with other excipients, to provide a solid pharmaceutical dosage form that contains both Compound 1 and Compound 2.

In still another embodiment, Compound 1 and Compound 2 are formulated in the same amorphous solid dispersion. The solid dispersion is milled and/or mixed with other excipients, and then compressed into a tablet.

In a yet another embodiment, a solid pharmaceutical composition of the invention comprises:

(1) Compound 1 or a pharmaceutically acceptable salt thereof, formulated in a first amorphous solid dispersion, wherein the first amorphous solid dispersion further comprises a pharmaceutically acceptable hydrophilic polymer and a pharmaceutically acceptable surfactant; and

(2) Compound 2 or a pharmaceutically acceptable salt thereof, formulated in a second amorphous solid dispersion, wherein the second amorphous solid dispersion further comprises a pharmaceutically acceptable hydrophilic polymer and a pharmaceutically acceptable surfactant.

In yet another embodiment, a solid pharmaceutical composition of the invention is a tablet which comprises:

(1) a first layer comprising a first amorphous solid dispersion, wherein the first amorphous solid dispersion comprises (i) Compound 1 or a pharmaceutically acceptable salt thereof, (ii) a pharmaceutically acceptable hydrophilic polymer and (iii) a pharmaceutically acceptable surfactant; and

(2) a second layer comprising a second amorphous solid dispersion, wherein the second amorphous solid dispersion comprises (i) Compound 2 or a pharmaceutically acceptable salt thereof, (ii) a pharmaceutically acceptable hydrophilic polymer and (iii) a pharmaceutically acceptable surfactant.

In a yet another embodiment, a solid pharmaceutical composition of the invention comprises:

(1) 100 mg of Compound 1 formulated in amorphous solid dispersion which further comprises a pharmaceutically acceptable hydrophilic polymer and a pharmaceutically acceptable surfactant; and

(2) 40 mg of Compound 2 formulated in amorphous solid dispersion which further comprises a pharmaceutically acceptable hydrophilic polymer and a pharmaceutically acceptable surfactant.

In a yet another embodiment, a solid pharmaceutical composition of the invention comprises:

(1) 100 mg of Compound 1 formulated in amorphous solid dispersion which further comprises copovidone and Vitamin E polyethylene glycol succinate (Vitamin E TPGS); and

(2) 40 mg of Compound 2 formulated in amorphous solid dispersion which further comprises copovidone and Vitamin E TPGS.

In a yet another embodiment, a solid pharmaceutical composition of the invention comprises:

(1) 100 mg of Compound 1 formulated in amorphous solid dispersion which further comprises copovidone and Vitamin E TPGS; and

(2) 40 mg of Compound 2 formulated in amorphous solid dispersion which further comprises copovidone, Vitamin E TPGS and propylene glycol monocaprylate.

In a yet another embodiment, a solid pharmaceutical composition of the invention comprises:

(1) 50 mg of Compound 1 formulated in amorphous solid dispersion which further comprises a pharmaceutically acceptable hydrophilic polymer and a pharmaceutically acceptable surfactant; and

(2) 20 mg of Compound 2 formulated in amorphous solid dispersion which further comprises a pharmaceutically acceptable hydrophilic polymer and a pharmaceutically acceptable surfactant.

In a yet another embodiment, a solid pharmaceutical composition of the invention comprises:

(1) 50 mg of Compound 1 formulated in amorphous solid dispersion which further comprises copovidone and Vitamin E polyethylene glycol succinate (Vitamin E TPGS); and

(2) 20 mg of Compound 2 formulated in amorphous solid dispersion which further comprises copovidone and Vitamin E TPGS.

In a yet another embodiment, a solid pharmaceutical composition of the invention comprises:

(1) 50 mg of Compound 1 formulated in amorphous solid dispersion which further comprises copovidone and Vitamin E TPGS; and

(2) 20 mg of Compound 2 formulated in amorphous solid dispersion which further comprises copovidone, Vitamin E TPGS and propylene glycol monocaprylate.

In yet another embodiment, a solid pharmaceutical composition of the invention is a tablet which comprises:

(1) a first layer which comprises 100 mg of Compound 1, as well as a pharmaceutically acceptable hydrophilic polymer and a pharmaceutically acceptable surfactant, all of which are formulated in amorphous solid dispersion; and

(2) a second layer which comprises 40 mg of Compound 2, as well as a pharmaceutically acceptable hydrophilic polymer and a pharmaceutically acceptable surfactant, all of which are formulated in amorphous solid dispersion.

In yet another embodiment, a solid pharmaceutical composition of the invention is a tablet which comprises:

(1) a first layer which comprises 100 mg of Compound 1, as well as copovidone and Vitamin E TPGS, all of which are formulated in amorphous solid dispersion; and

(2) a second layer which comprises 40 mg of Compound 2, as well as copovidone and Vitamin E TPGS, all of which are formulated in amorphous solid dispersion.

In yet another embodiment, a solid pharmaceutical composition of the invention is a tablet which comprises:

(1) a first layer which comprises 100 mg of Compound 1, as well as copovidone and Vitamin E TPGS, all of which are formulated in amorphous solid dispersion; and

(2) a second layer which comprises 40 mg of Compound 2, as well as copovidone, Vitamin E TPGS and propylene glycol monocaprylate, all of which are formulated in amorphous solid dispersion.

In a yet another embodiment, a solid pharmaceutical composition of the invention comprises:

(1) a first type of mini-tablets, each of which is no more than 5 mm in size and comprises an amorphous solid dispersion including (i) Compound 1 or a pharmaceutically acceptable salt thereof, (ii) a pharmaceutically acceptable hydrophilic polymer and (iii) a pharmaceutically acceptable surfactant; and

(2) a second type of mini-tablets, each of which is no more than 5 mm in size and comprises an amorphous solid dispersion including (i) Compound 2 or a pharmaceutically acceptable salt thereof, (ii) a pharmaceutically acceptable hydrophilic polymer and (iii) a pharmaceutically acceptable surfactant.

In a yet another embodiment, a solid pharmaceutical composition of the invention comprises:

(1) a first type of mini-tablets, each of which is no more than 3 mm in size and comprises an amorphous solid dispersion including (i) Compound 1 or a pharmaceutically acceptable salt thereof, (ii) a pharmaceutically acceptable hydrophilic polymer and (iii) a pharmaceutically acceptable surfactant; and

(2) a second type of mini-tablets, each of which is no more than 3 mm in size and comprises an amorphous solid dispersion including (i) Compound 2 or a pharmaceutically acceptable salt thereof, (ii) a pharmaceutically acceptable hydrophilic polymer and (iii) a pharmaceutically acceptable surfactant.

In a yet another embodiment, a solid pharmaceutical composition of the invention comprises:

(1) a first type of mini-tablets, each of which is no more than 2 mm in size and comprises an amorphous solid dispersion including (i) Compound 1 or a pharmaceutically acceptable salt thereof, (ii) a pharmaceutically acceptable hydrophilic polymer and (iii) a pharmaceutically acceptable surfactant; and

(2) a second type of mini-tablets, each of which is no more than 2 mm in size and comprises an amorphous solid dispersion including (i) Compound 2 or a pharmaceutically acceptable salt thereof, (ii) a pharmaceutically acceptable hydrophilic polymer and (iii) a pharmaceutically acceptable surfactant.

In a yet another embodiment, a solid pharmaceutical composition of the invention comprises:

(1) a first type of mini-tablets, each of which is no more than 5 mm in size and comprises an amorphous solid dispersion including (i) Compound 1, (ii) a pharmaceutically acceptable hydrophilic polymer and (iii) a pharmaceutically acceptable surfactant, and wherein the total amount of Compound 1 comprised in the first type of mini-tablets is 100 mg; and

(2) a second type of mini-tablets, each of which is no more than 5 mm in size and comprises an amorphous solid dispersion including (i) Compound 2, (ii) a pharmaceutically acceptable hydrophilic polymer and (iii) a pharmaceutically acceptable surfactant, and wherein the total amount of Compound 2 comprised in the second type of mini-tablets is 40 mg.

In a yet another embodiment, a solid pharmaceutical composition of the invention comprises:

(1) a first type of mini-tablets, each of which is no more than 3 mm in size and comprises an amorphous solid dispersion including (i) Compound 1, (ii) a pharmaceutically acceptable hydrophilic polymer and (iii) a pharmaceutically acceptable surfactant, and wherein the total amount of Compound 1 comprised in the first type of mini-tablets is 100 mg; and

(2) a second type of mini-tablets, each of which is no more than 3 mm in size and comprises an amorphous solid dispersion including (i) Compound 2, (ii) a pharmaceutically acceptable hydrophilic polymer and (iii) a pharmaceutically acceptable surfactant, and wherein the total amount of Compound 2 comprised in the second type of mini-tablets is 40 mg.

In a yet another embodiment, a solid pharmaceutical composition of the invention comprises:

(1) a first type of mini-tablets, each of which is no more than 2 mm in size and comprises an amorphous solid dispersion including (i) Compound 1, (ii) a pharmaceutically acceptable hydrophilic polymer and (iii) a pharmaceutically acceptable surfactant, and wherein the total amount of Compound 1 comprised in the first type of mini-tablets is 100 mg; and

(2) a second type of mini-tablets, each of which is no more than 2 mm in size and comprises an amorphous solid dispersion including (i) Compound 2, (ii) a pharmaceutically acceptable hydrophilic polymer and (iii) a pharmaceutically acceptable surfactant, and wherein the total amount of Compound 2 comprised in the second type of mini-tablets is 40 mg.

In a yet another embodiment, a solid pharmaceutical composition of the invention comprises:

(1) a first type of mini-tablets, each of which is no more than 5 mm in size and comprises an amorphous solid dispersion including (i) Compound 1, (ii) copovidone and (iii) Vitamin E TPGS, and wherein the total amount of Compound 1 comprised in the first type of mini-tablets is 100 mg; and

(2) a second type of mini-tablets, each of which is no more than 5 mm in size and comprises an amorphous solid dispersion including (i) Compound 2, (ii) copovidone and (iii) Vitamin E TPGS, and wherein the total amount of Compound 2 comprised in the second type of mini-tablets is 40 mg.

In a yet another embodiment, a solid pharmaceutical composition of the invention comprises:

(1) a first type of mini-tablets, each of which is no more than 3 mm in size and comprises an amorphous solid dispersion including (i) Compound 1, (ii) copovidone and (iii) Vitamin E TPGS, and wherein the total amount of Compound 1 comprised in the first type of mini-tablets is 100 mg; and

(2) a second type of mini-tablets, each of which is no more than 3 mm in size and comprises an amorphous solid dispersion including (i) Compound 2, (ii) copovidone and (iii) Vitamin E TPGS, and wherein the total amount of Compound 2 comprised in the second type of mini-tablets is 40 mg.

In a yet another embodiment, a solid pharmaceutical composition of the invention comprises:

(1) a first type of mini-tablets, each of which is no more than 2 mm in size and comprises an amorphous solid dispersion including (i) Compound 1, (ii) copovidone and (iii) Vitamin E TPGS, and wherein the total amount of Compound 1 comprised in the first type of mini-tablets is 100 mg; and

(2) a second type of mini-tablets, each of which is no more than 2 mm in size and comprises an amorphous solid dispersion including (i) Compound 2, (ii) copovidone and (iii) Vitamin E TPGS, and wherein the total amount of Compound 2 comprised in the second type of mini-tablets is 40 mg.

In a yet another embodiment, a solid pharmaceutical composition of the invention comprises:

(1) a first type of mini-tablets, each of which is no more than 5 mm in size and comprises an amorphous solid dispersion including (i) Compound 1, (ii) copovidone and (iii) Vitamin E TPGS, and wherein the total amount of Compound 1 comprised in the first type of mini-tablets is 100 mg; and

(2) a second type of mini-tablets, each of which is no more than 5 mm in size and comprises an amorphous solid dispersion including (i) Compound 2, (ii) copovidone and (iii) Vitamin E TPGS and propylene glycol monocaprylate, and wherein the total amount of Compound 2 comprised in the second type of mini-tablets is 40 mg.

In a yet another embodiment, a solid pharmaceutical composition of the invention comprises:

(1) a first type of mini-tablets, each of which is no more than 3 mm in size and comprises an amorphous solid dispersion including (i) Compound 1, (ii) copovidone and (iii) Vitamin E TPGS, and wherein the total amount of Compound 1 comprised in the first type of mini-tablets is 100 mg; and

(2) a second type of mini-tablets, each of which is no more than 3 mm in size and comprises an amorphous solid dispersion including (i) Compound 2, (ii) copovidone and (iii) Vitamin E TPGS and propylene glycol monocaprylate, and wherein the total amount of Compound 2 comprised in the second type of mini-tablets is 40 mg.

In a yet another embodiment, a solid pharmaceutical composition of the invention comprises:

(1) a first type of mini-tablets, each of which is no more than 2 mm in size and comprises an amorphous solid dispersion including (i) Compound 1, (ii) copovidone and (iii) Vitamin E TPGS, and wherein the total amount of Compound 1 comprised in the first type of mini-tablets is 100 mg; and

(2) a second type of mini-tablets, each of which is no more than 2 mm in size and comprises an amorphous solid dispersion including (i) Compound 2, (ii) copovidone and (iii) Vitamin E TPGS and propylene glycol monocaprylate, and wherein the total amount of Compound 2 comprised in the second type of mini-tablets is 40 mg.

In a yet another embodiment, a solid pharmaceutical composition of the invention comprises:

(1) a first type of film-coated granules comprising an amorphous solid dispersion including (i) Compound 1, (ii) copovidone and (iii) Vitamin E TPGS, and wherein the total amount of Compound 1 comprised in the first type of granules is 50 mg; and

(2) a second type of film-coated granules comprising an amorphous solid dispersion including (i) Compound 2, (ii) copovidone and (iii) Vitamin E TPGS and propylene glycol monocaprylate, and wherein the total amount of Compound 2 comprised in the second type of granules is 20 mg. In some such embodiments, the first type of film-coated granules and/or the second type of film-coated granules are contained in a dispensing container such as a sachet.

Preferably, in any aspect, embodiment, example, preference and composition of the invention, the total weight of Compound 1 in amorphous solid dispersion ranges from 10% to 40% by weight relative to the total weight of the amorphous solid dispersion. More preferably, in any aspect, embodiment, example, preference and composition of the invention, the total weight of Compound 1 in amorphous solid dispersion ranges from 15% to 30% by weight relative to the total weight of the amorphous solid dispersion. Highly preferably, in any aspect, embodiment, example, preference and composition of the invention, the total weight of Compound 1 in amorphous solid dispersion is 20% by weight relative to the total weight of the amorphous solid dispersion.

Preferably, in any aspect, embodiment, example, preference and composition of the invention, the total weight of Compound 2 in amorphous solid dispersion ranges from 5% to 20% by weight relative to the total weight of the amorphous solid dispersion. More preferably, in any aspect, embodiment, example, preference and composition of the invention, the total weight of Compound 2 in amorphous solid dispersion is 10% by weight relative to the total weight of the amorphous solid dispersion.

More preferably, in any aspect, embodiment, example, preference and composition of the invention, the total weight of Compound 1 in amorphous solid dispersion ranges from 15% to 30% by weight relative to the total weight of the amorphous solid dispersion. And the total weight of Compound 2 in amorphous solid dispersion ranges from 5% to 15% by weight relative to the total weight of the amorphous solid dispersion.

Highly preferably, in any aspect, embodiment, example, preference and composition of the invention, the total weight of Compound 1 in amorphous solid dispersion is 20% by weight relative to the total weight of the amorphous solid dispersion. And the total weight of Compound 2 in amorphous solid dispersion is 10% by weight relative to the total weight of the amorphous solid dispersion.

Preferably, in any aspect, embodiment, example, preference and composition of the invention, the amorphous solid dispersion can comprise from 50% to 80% by weight, relative to the total weight of the amorphous solid dispersion, of a pharmaceutically acceptable hydrophilic polymer, and from 5% to 15% by weight, relative to the total weight of the amorphous solid dispersion, of a pharmaceutically acceptable surfactant.

Preferably, in any aspect, embodiment, example, preference and composition of the invention, the amorphous solid dispersion can comprise from 50% to 90% by weight, relative to the total weight of the amorphous solid dispersion, of a pharmaceutically acceptable hydrophilic polymer, and from 5% to 15% by weight, relative to the total weight of the amorphous solid dispersion, of a pharmaceutically acceptable surfactant.

Also preferably, in any aspect, embodiment, example, preference and composition of the invention, the amorphous solid dispersion can comprise from 60% to 80% by weight, relative to the total weight of the amorphous solid dispersion, of a pharmaceutically acceptable hydrophilic polymer, and 10% by weight, relative to the total weight of the amorphous solid dispersion, of a pharmaceutically acceptable surfactant.

In any aspect, embodiment, example, preference and composition of the invention, the pharmaceutically acceptable hydrophilic polymer can have a T_{g} of at least 50° C.; preferably, the pharmaceutically acceptable hydrophilic polymer has a T_{g} of at least 80° C.; more preferably, the pharmaceutically acceptable hydrophilic polymer has a T_{g} of at least 100° C. For example, the pharmaceutically acceptable hydrophilic polymer can have a T_{g} of from 80° C. to 180° C., or from 100° C. to 150° C.

Preferably, the pharmaceutically acceptable hydrophilic polymer employed in the present invention is water-soluble. A solid pharmaceutical composition of the invention can also comprise poorly water-soluble or water-insoluble polymers, such as cross-linked polymers. The pharmaceutically acceptable hydrophilic polymer comprised in a solid pharmaceutical composition of the invention preferably has an apparent viscosity, when dissolved at 20° C. in an aqueous solution at 2% (w/v), of 1 to 5000 mPa·s., and more preferably of 1 to 700 mPa·s, and most preferably of 5 to 100 mPa·s.

In any aspect, embodiment, example and composition of the invention, the pharmaceutically acceptable hydrophilic polymer can be selected from homopolymer of N-vinyl lactam, copolymer of N-vinyl lactam, cellulose ester, cellulose ether, polyalkylene oxide, polyacrylate, polymethacrylate, polyacrylamide, polyvinyl alcohol, vinyl acetate polymer, oligosaccharide, polysaccharide, or combinations thereof. Non-limiting examples of suitable hydrophilic polymers include homopolymer of N-vinyl pyrrolidone, copolymer of N-vinyl pyrrolidone, copolymer of N-vinyl pyrrolidone and vinyl acetate, copolymer of N-vinyl pyrrolidone and vinyl propionate, polyvinylpyrrolidone, methylcellulose, ethylcellulose, hydroxyalkylcelluloses, hydroxypropylcellulose, hydroxyalkylalkylcellulose, hydroxypropylmethylcellulose, cellulose phthalate, cellulose succinate, cellulose acetate phthalate, hydroxypropylmethylcellulose phthalate, hydroxypropylmethylcellulose succinate, hydroxypropylmethylcellulose acetate succinate, polyethylene oxide, polypropylene oxide, copolymer of ethylene oxide and propylene oxide, methacrylic acid/ethyl acrylate copolymer, methacrylic acid/methyl methacrylate copolymer, butyl methacrylate/2-dimethylaminoethyl methacrylate copolymer, poly(hydroxyalkyl acrylate), poly(hydroxyalkyl methacrylate), copolymer of vinyl acetate and crotonic acid, partially hydrolyzed polyvinyl acetate, carrageenan, galactomannan, xanthan gum, or combinations thereof.

Preferably, in any aspect, embodiment, example, preference and composition of the invention, the polymer is copovidone.

In any aspect, embodiment, example, preference and composition of the invention, the pharmaceutically acceptable surfactant can have an HLB value of at least 10. Surfactants having an HLB value of less than 10 can also be used.

In any aspect, embodiment, example, preference and composition of the invention, the pharmaceutically acceptable surfactant can be selected from polyoxyethylene castor oil derivates, mono fatty acid ester of polyoxyethylene sorbitan, polyoxyethylene alkyl ether, polyoxyethylene alkylaryl ether, polyethylene glycol fatty acid ester, alkylene glycol fatty acid mono ester, sucrose fatty acid ester, sorbitan fatty acid mono ester, or combinations thereof. Non-limiting examples of suitable surfactants include polyoxyethyleneglycerol triricinoleate or polyoxyl 35 castor oil (Cremophor® EL; BASF Corp.) or polyoxyethyleneglycerol oxystearate such as polyethylenglycol 40 hydrogenated castor oil (Cremophor® RH 40, also known as polyoxyl 40 hydrogenated castor oil or macrogolglycerol hydroxystearate) or polyethylenglycol 60 hydrogenated castor oil (Cremophor® RH 60), mono fatty acid ester of polyoxyethylene sorbitan, such as mono fatty acid ester of polyoxyethylene (20) sorbitan, e.g. polyoxyethylene (20) sorbitan monooleate (Tween® 80), polyoxyethylene (20) sorbitan monostearate (Tween® 60), polyoxyethylene (20) sorbitan monopalmitate (Tween® 40) or polyoxyethylene (20) sorbitan monolaurate (Tween® 20), polyoxyethylene (3) lauryl ether, polyoxyethylene (5) cetyl ether, polyoxyethylene (2) stearyl ether, polyoxyethylene (5) stearyl ether, polyoxyethylene (2) nonylphenyl ether, polyoxyethylene (3) nonylphenyl ether, polyoxyethylene (4) nonylphenyl ether, polyoxyethylene (3) octylphenyl ether, PEG-200 monolaurate, PEG-200 dilaurate, PEG-300 dilaurate, PEG-400 dilaurate, PEG-300 distearate, PEG-300 dioleate, propylene glycol monolaurate (e.g., Lauroglycol), sucrose monostearate, sucrose distearate, sucrose monolaurate, sucrose dilaurate, sorbitan mono laurate, sorbitan monooleate, sorbitan monopalnitate, sorbitan stearate, or combinations thereof.

Preferably, in any aspect, embodiment, example, preference and composition of the invention, the pharmaceutically acceptable surfactant is or includes D-alpha-tocopheryl polyethylene glycol 1000 succinate (vitamin E TPGS).

Also preferably, in any aspect, embodiment, example, preference and composition of the invention, the pharmaceutically acceptable surfactant used in the amorphous solid dispersion comprising Compound 2 is or includes a combination of Vitamin E TPGS and propylene glycol monocaprylate.

Highly preferably, in any aspect, embodiment, example, preference and composition of the invention, the pharmaceutically acceptable hydrophilic polymer is copovidone, and the pharmaceutically acceptable surfactant is or includes vitamin E TPGS.

In any aspect, embodiment, example, preference and composition of the invention, the amorphous solid dispersion preferably comprises or consists of a single-phase (defined in thermodynamics) in which Compound 1 or Compound 2 is amorphously dispersed in a matrix containing the pharmaceutically acceptable hydrophilic polymer and the pharmaceutically acceptable surfactant. Thermal analysis of the amorphous solid dispersion using differential scanning calorimetry (DSC) typically shows only one single T_{g}, and the amorphous solid dispersion typically does not contain any detectable crystalline compound as measured by X-ray powder diffraction spectroscopy.

In any aspect, embodiment, example, preference and composition of the invention, the solid pharmaceutical composition of the invention can be a tablet.

In any aspect, embodiment, example, preference and composition of the invention, the solid pharmaceutical composition of the invention can be a mixture of mini-tablets.

In any aspect, embodiment, example, preference and composition of the invention, the solid pharmaceutical composition of the invention can be a mixture of granules, which may be contained in a dispensing container such as a packet or sachet.

In any aspect, embodiment, example, preference and composition of the invention, the solid pharmaceutical composition of the invention can be prepared into other suitable dosage forms, such as capsule, dragee, granule, or powder.

In any aspect, embodiment, example, preference and composition of the invention, the solid pharmaceutical composition of the invention is administered to a HCV patient with food to treat HCV. Administration with food can significantly improve the bioavailability of Compound 1 and Compound 2 in the patient when delivered using the solid pharmaceutical composition of the invention.

A solid pharmaceutical composition of the invention can further comprise another anti-HCV agent, for example, an agent selected from HCV helicase inhibitors, HCV polymerase inhibitors, HCV protease inhibitors, HCV NS5A inhibitors, CD81 inhibitors, cyclophilin inhibitors, or internal ribosome entry site (IBES) inhibitors.

In one embodiment, the invention provides a stable, oral, immediate release solid pharmaceutical composition comprising:
(1) a first type of film-coated granule comprising an amorphous solid dispersion including (i) 50 mg of Compound 1, (ii) 50% to 80% by weight of a first pharmaceutically acceptable polymer and (iii) from 5% to 15% by weight of a first pharmaceutically acceptable surfactant; and
(2) a second type of film-coated granule comprising an amorphous solid dispersion including (i) 20 mg of Compound 2, (ii) 50% to 90% by weight of a second pharmaceutically acceptable polymer and (iii) from 5% to 15% by weight of a second pharmaceutically acceptable surfactant.

In one embodiment, the invention provides a stable, oral, immediate release solid pharmaceutical composition comprising:
(1) a first type of film-coated granule comprising an amorphous solid dispersion including (i) 50 mg of Compound 1, (ii) 50% to 80% by weight of copovidone and (iii) from 5% to 15% by weight of Vitamin E TPGS; and
(2) a second type of film-coated granule comprising an amorphous solid dispersion including (i) 20 mg of Compound 2, (ii) 50% to 90% by weight of copovidone and (iii) from 5% to 15% by weight of Vitamin E TPGS and propylene glycol monocaprylate.

In one embodiment, the invention provides a stable, oral, immediate release solid pharmaceutical composition comprising:
(1) a first type of film-coated granule comprising an amorphous solid dispersion including (i) 50 mg of Compound 1, (ii) 50% to 80% by weight of a first pharmaceutically acceptable polymer and (iii) from 5% to 15% by weight of a first pharmaceutically acceptable surfactant; and
(2) a second type of film-coated granule comprising an amorphous solid dispersion including (i) 20 mg of Compound 2, (ii) 50% to 90% by weight of a second pharmaceutically acceptable polymer and (iii) from 5% to 15% by weight of a second pharmaceutically acceptable surfactant,
   wherein the composition is provided in a sachet and is stable for the duration of a shelf life of about 24 months in the sachet.

In one embodiment, the invention provides a stable, oral, immediate release solid pharmaceutical composition comprising:
(1) a first type of film-coated granule comprising an amorphous solid dispersion including (i) 50 mg of Compound 1, (ii) 50% to 80% by weight of copovidone and (iii) from 5% to 15% by weight of Vitamin E TPGS; and
(2) a second type of film-coated granule comprising an amorphous solid dispersion including (i) 20 mg of Compound 2, (ii) 50% to 90% by weight of copovidone and (iii) from 5% to 15% by weight of Vitamin E TPGS and propylene glycol monocaprylate,
   wherein the composition is provided in a sachet and is stable for the duration of a shelf life of about 24 months in the sachet.

In one embodiment, the invention provides a stable, oral, immediate release solid pharmaceutical composition comprising:
(1) a first type of film-coated granule comprising an amorphous solid dispersion including (i) 50 mg of Compound 1, (ii) about 172.5 mg of copovidone and (iii) about 25 mg of Vitamin E TPGS; and
(2) a second type of film-coated granule comprising an amorphous solid dispersion including (i) 20 mg of Compound 2, (ii) about 158.0 mg of copovidone and (iii) about 16.0 mg of Vitamin E TPGS and about 4.0 mg or propylene glycol monocaprylate.

In one embodiment, the invention provides a stable, oral, immediate release solid pharmaceutical composition comprising:
(1) a first type of film-coated granule comprising an amorphous solid dispersion including (i) 50 mg of Compound 1, (ii) about 172.5 mg of copovidone and (iii) about 25 mg of Vitamin E TPGS; and
(2) a second type of film-coated granule comprising an amorphous solid dispersion including (i) 20 mg of Compound 2, (ii) about 158.0 mg of copovidone and (iii) about 16.0 mg of Vitamin E TPGS and about 4.0 mg or propylene glycol monocaprylate,

wherein the composition is provided in a sachet and is stable for the duration of a shelf life of about 24 months in the sachet.

Any composition of the invention, as described or contemplated herein (e.g., the compositions described in Examples 1 and 2), preferably has the following in vitro release profile: when dissolved in 1000 mL of a dissolution medium using a standard USP dissolution Apparatus 2 (paddle) with Japanese sinker operating at 75 RPM at 37° C., at least 80% of Compound 1 in the composition is released within 3 hours and at least 80% of Compound 2 in the composition is released within 3 hours, wherein the dissolution medium is 0.1 M Acetate buffer (pH 4.0) with 1% Polysorbate 80.

Any composition of the invention, as described or contemplated herein (e.g., the compositions described in Examples 1 and 2), preferably has the following in vitro release profile: when the composition is dissolved in 500 mL of a dissolution medium using a standard USP dissolution Apparatus 1 (basket) operating at 75 RPM at 37° C., at least 80% of Compound 1 in the composition is released within 40 minutes and at least 80% of Compound 2 in the composition is released within 40 minutes, wherein the dissolution medium is 0.1 M Acetate buffer (pH 4.0) with 1% Polysorbate 80.

Any composition of the invention, as described or contemplated herein (e.g., the compositions described in Examples 1 and 2), preferably has the following in vitro release profile: when dissolved in 1000 mL of a dissolution medium using a standard USP dissolution Apparatus 2 (paddle) with Japanese sinker operating at 75 RPM at 37° C., at least 90% of Compound 1 in the composition is released within 3 hours and at least 90% of Compound 2 in the composition is released within 3 hours, wherein the dissolution medium is 0.1 M Acetate buffer (pH 4.0) with 1% Polysorbate 80.

Any composition of the invention, as described or contemplated herein (e.g., the compositions described in Examples 1 and 2), preferably has the following in vitro release profile: when dissolved in 1000 mL of a dissolution medium using a standard USP dissolution Apparatus 2 (paddle) with Japanese sinker operating at 75 RPM at 37° C., at least 75% of Compound 1 in the composition is released within 105 minutes and at least 80% of Compound 2 in the composition is released within 105 minutes, wherein the dissolution medium is 0.1 M Acetate buffer (pH 4.0) with 1% Polysorbate 80.

Any composition of the invention, as described or contemplated herein (e.g., the compositions described in Examples 1 and 2), preferably has the following in vitro release profile: when dissolved in 1000 mL of a dissolution medium using a standard USP dissolution Apparatus 2 (paddle) with Japanese sinker operating at 75 RPM at 37° C., at least 80% of Compound 1 in the composition is released within 100 minutes and at least 80% of Compound 2 in the composition is released within 100 minutes, wherein the dissolution medium is 0.1 M Acetate buffer (pH 4.0) with 1% Polysorbate 80.

Any composition of the invention, as described or contemplated herein (e.g., the compositions described in Examples 1 and 2), preferably has the following in vitro release profile: when dissolved in 1000 mL of a dissolution medium using a standard USP dissolution Apparatus 2 (paddle) with Japanese sinker operating at 75 RPM at 37° C., at least 40% of Compound 1 in the composition is released within 50 minutes and at least 50% of Compound 2 in the composition is released within 50 minutes, wherein the dissolution medium is 0.1 M Acetate buffer (pH 4.0) with 1% Polysorbate 80.

Any composition of the invention, as described or contemplated herein (e.g., the compositions described in Examples 1 and 2), preferably has the following in vitro release profile: when dissolved in 1000 mL of a dissolution medium using a standard USP dissolution Apparatus 2 (paddle) with Japanese sinker operating at 75 RPM at 37° C., at least 30% of Compound 1 in the composition is released within 50 minutes and at least 45% of Compound 2 in the composition is released within 50 minutes, wherein the dissolution medium is 0.1 M Acetate buffer (pH 4.0) with 1% Polysorbate 80.

Any composition of the invention, as described or contemplated herein (e.g., the compositions described in Examples 1 and 2), preferably has the following in vitro release profile: when the composition is dissolved in 500 mL of a dissolution medium using a standard USP dissolution Apparatus 1 (basket) operating at 75 RPM at 37° C., at least 30% of Compound 1 in the composition is released within 20 minutes and at least 45% of Compound 2 in the composition is released within 20 minutes, wherein the dissolution medium is 0.1 M Acetate buffer (pH 4.0) with 1% Polysorbate 80.

Any composition of the invention, as described or contemplated herein (e.g., the compositions described in Examples 1 and 2), preferably has the following in vitro release profile: when dissolved in 1000 mL of a dissolution medium using a standard USP dissolution Apparatus 2 (paddle) with Japanese sinker operating at 75 RPM at 37° C., at least 10% of Compound 1 in the composition is released within 25 minutes and at least 20% of Compound 2 in the composition is released within 25 minutes, wherein the dissolution medium is 0.1 M Acetate buffer (pH 4.0) with 1% Polysorbate 80.

Any composition of the invention, as described or contemplated herein (e.g., the compositions described in Examples 1 and 2), preferably has the following in vitro release profile: when dissolved in 1000 mL of a dissolution medium using a standard USP dissolution Apparatus 2 (paddle) with Japanese sinker operating at 75 RPM at 37° C., at least 5% of Compound 1 in the composition is released within 25 minutes and at least 10% of Compound 2 in the composition is released within 25 minutes, wherein the dissolution medium is 0.1 M Acetate buffer (pH 4.0) with 1% Polysorbate 80.

Any composition of the invention, as described or contemplated herein (e.g., the compositions described in Examples 1 and 2), preferably has the following in vitro release profile: when the composition is dissolved in 500 mL of a dissolution medium using a standard USP dissolution Apparatus 1 (basket) operating at 75 RPM at 37° C., at least 5% of Compound 1 in the composition is released within 10 minutes and at least 10% of Compound 2 in the composition is released within 10 minutes, wherein the dissolution medium is 0.1 M Acetate buffer (pH 4.0) with 1% Polysorbate 80.

Any composition of the invention, as described or contemplated herein (e.g., the compositions described in Examples 1 and 2), preferably has the following in vitro release profile: when dissolved in 1000 mL of a dissolution medium using a standard USP dissolution Apparatus 2 (paddle) with Japanese sinker operating at 75 RPM at 37° C., 80-100% of Compound 1 in the composition is released within 3 hours and at least 80-100% of Compound 2 in the composition is released within 3 hours, wherein the dissolution medium is 0.1 M Acetate buffer (pH 4.0) with 1% Polysorbate 80.

Any composition of the invention, as described or contemplated herein (e.g., the compositions described in Examples 1 and 2), preferably has the following in vitro release profile: when dissolved in 1000 mL of a dissolution medium using a standard USP dissolution Apparatus 2 (paddle) with Japanese sinker operating at 75 RPM at 37° C., 90-100% of Compound 1 in the composition is released within 3 hours and at least 90-100% of Compound 2 in the composition is released within 3 hours, wherein the dissolution medium is 0.1 M Acetate buffer (pH 4.0) with 1% Polysorbate 80.

Any composition of the invention, as described or contemplated herein (e.g., the compositions described in Examples 1 and 2), preferably has the following in vitro release profile: when dissolved in 1000 mL of a dissolution medium using a standard USP dissolution Apparatus 2 (paddle) with Japanese sinker operating at 75 RPM at 37° C., 75-100% of Compound 1 in the composition is released within 105 minutes and 80-100% of Compound 2 in the composition is released within 105 minutes, wherein the dissolution medium is 0.1 M Acetate buffer (pH 4.0) with 1% Polysorbate 80.

Any composition of the invention, as described or contemplated herein (e.g., the compositions described in Examples 1 and 2), preferably has the following in vitro release profile: when dissolved in 1000 mL of a dissolution medium using a standard USP dissolution Apparatus 2 (paddle) with Japanese sinker operating at 75 RPM at 37° C., 80-100% of Compound 1 in the composition is released within 100 minutes and 85-100% of Compound 2 in the composition is released within 100 minutes, wherein the dissolution medium is 0.1 M Acetate buffer (pH 4.0) with 1% Polysorbate 80.

Any composition of the invention, as described or contemplated herein (e.g., the compositions described in Examples 1 and 2), preferably has the following in vitro release profile: when dissolved in 1000 mL of a dissolution medium using a standard USP dissolution Apparatus 2 (paddle) with Japanese sinker operating at 75 RPM at 37° C., 40-60% of Compound 1 in the composition is released within 50 minutes and 50-80% of Compound 2 in the composition is released within 50 minutes, wherein the dissolution medium is 0.1 M Acetate buffer (pH 4.0) with 1% Polysorbate 80.

Any composition of the invention, as described or contemplated herein (e.g., the compositions described in Examples 1 and 2), preferably has the following in vitro release profile: when dissolved in 1000 mL of a dissolution medium using a standard USP dissolution Apparatus 2 (paddle) with Japanese sinker operating at 75 RPM at 37° C., 30-60% of Compound 1 in the composition is released within 50 minutes and 45-80% of Compound 2 in the composition is released within 50 minutes, wherein the dissolution medium is 0.1 M Acetate buffer (pH 4.0) with 1% Polysorbate 80.

Any composition of the invention, as described or contemplated herein (e.g., the compositions described in Examples 1 and 2), preferably has the following in vitro release profile: when dissolved in 1000 mL of a dissolution medium using a standard USP dissolution Apparatus 2 (paddle) with Japanese sinker operating at 75 RPM at 37° C., 10-30% of Compound 1 in the composition is released within 25 minutes and 20-40% of Compound 2 in the composition is released within 25 minutes, wherein the dissolution medium is 0.1 M Acetate buffer (pH 4.0) with 1% Polysorbate 80.

Any composition of the invention, as described or contemplated herein (e.g., the compositions described in Examples 1 and 2), preferably has the following in vitro release profile: when dissolved in 1000 mL of a dissolution medium using a standard USP dissolution Apparatus 2 (paddle) with Japanese sinker operating at 75 RPM at 37° C., 5-30% of Compound 1 in the composition is released within 25 minutes and 10-40% of Compound 2 in the composition is released within 25 minutes, wherein the dissolution medium is 0.1 M Acetate buffer (pH 4.0) with 1% Polysorbate 80.

Any composition of the invention, as described or contemplated herein (e.g., the compositions described in Examples 1 and 2), preferably has the following in vitro release profile: when dissolved in 1000 mL of a dissolution medium using a standard USP dissolution Apparatus 2 (paddle) with Japanese sinker operating at 75 RPM at 37° C., 10-30% of Compound 1 in the composition is released within 25 minutes and 20-40% of Compound 2 in the composition is released within 25 minutes, 40-60% of Compound 1 in the composition is released within 50 minutes and 50-80% of Compound 2 in the composition is released within 50 minutes, 80-100% of Compound 1 in the composition is released within 100 minutes and 85-100% of Compound 2 in the composition is released within 100 minutes, wherein the dissolution medium is 0.1 M Acetate buffer (pH 4.0) with 1% Polysorbate 80.

Any composition of the invention, as described or contemplated herein (e.g., the compositions described in Examples 1 and 2), preferably has the following in vitro release profile: when dissolved in 1000 mL of a dissolution medium using a standard USP dissolution Apparatus 2 (paddle) with Japanese sinker operating at 75 RPM at 37° C., 5-30% of Compound 1 in the composition is released within 25 minutes and 10-40% of Compound 2 in the composition is released within 25 minutes, 30-60% of Compound 1 in the composition is released within 50 minutes and 45-80% of Compound 2 in the composition is released within 50 minutes, 75-100% of Compound 1 in the composition is released within 105 minutes and 80-100% of Compound 2 in the composition is released within 105 minutes, wherein the dissolution medium is 0.1 M Acetate buffer (pH 4.0) with 1% Polysorbate 80.

Any composition of the invention, as described or contemplated herein (e.g., the compositions described in Examples 1 and 2), preferably has the following in vitro release profile: when the composition is dissolved in 500 mL of a dissolution medium using a standard USP dissolution Apparatus 1 (basket) operating at 75 RPM at 37° C., 80-100% of Compound 1 in the composition is released within 40 minutes and 80-100% of Compound 2 in the composition is released within 40 minutes, wherein the dissolution medium is 0.1 M Acetate buffer (pH 4.0) with 1% Polysorbate 80.

Any composition of the invention, as described or contemplated herein (e.g., the compositions described in Examples 1 and 2), preferably has the following in vitro release profile: when the composition is dissolved in 500 mL of a dissolution medium using a standard USP dissolution Apparatus 1 (basket) operating at 75 RPM at 37° C., 30-60% of Compound 1 in the composition is released within 20 minutes and 45-80% of Compound 2 in the composition is released within 20 minutes, wherein the dissolution medium is 0.1 M Acetate buffer (pH 4.0) with 1% Polysorbate 80.

Any composition of the invention, as described or contemplated herein (e.g., the compositions described in Examples 1 and 2), preferably has the following in vitro release profile: when the composition is dissolved in 500 mL of a dissolution medium using a standard USP dissolution Apparatus 1 (basket) operating at 75 RPM at 37° C., 5-25% of Compound 1 in the composition is released within 10 minutes and 10-30% of Compound 2 in the composition is released within 10 minutes, wherein the dissolution medium is 0.1 M Acetate buffer (pH 4.0) with 1% Polysorbate 80.

In another aspect, the present invention provides a composition that is bioequivalent to the solid pharmaceutical composition described herein. In some embodiments, the composition is bioequivalent according to its dissolution profile. In some embodiments, the composition is bioequivalent according to its bioavailability profile. For example, the bioequivalent composition may have an AUC value that is about 80% to about 125% of the AUC value of the solid pharmaceutical composition described herein. In other examples, the bioequivalent composition may have a Cₘₐₓ value that is about 80% to about 125% of the Cₘₐₓ value of the solid pharmaceutical composition described herein.

In another aspect, the solid pharmaceutical composition described herein is stable during its shelf life. In some embodiments, the composition has a shelf life of about 24 months in the sachet. In some embodiments, the composition has a shelf life of about 12 months in the sachet. In some embodiments, the composition has a shelf life of about 36 months in the sachet. In some embodiments, the composition has a shelf life of about 18 months in the sachet. In some embodiments, the composition has a shelf-life of about 6 months in the sachet. In some embodiments, the composition has a shelf life of about 6 months to about 36 months in the sachet. In some embodiments, the composition has a shelf life of about 6 months to about 30 months in the sachet. In some embodiments, the composition has a shelf life of about 6 months to about 24 months in the sachet. In some embodiments, the composition has a shelf life of about 6 months to about 18 months in the sachet. In some embodiments, the composition has a shelf life of about 6 months to about 12 months in the sachet. In some embodiments, the composition has a shelf life of up to about 6 months in the sachet. In some embodiments, the composition has a shelf life of up to about 12 months in the sachet. In some embodiments, the composition has a shelf life of up to about 18 months in the sachet. In some embodiments, the composition has a shelf life of up to about 24 months in the sachet. In some embodiments, the composition has a shelf life of up to about 30 months in the sachet. In some embodiments, the composition has a shelf life of up to about 36 months in the sachet.

In another aspect, the present invention features processes of making a solid pharmaceutical composition of the invention. The processes comprise (1) preparing a melt comprising a compound of interest, a pharmaceutically acceptable hydrophilic polymer, and a pharmaceutically acceptable surfactant; and (2) solidifying said melt. The solidified melt can comprise any amorphous solid dispersion described or contemplated herein. As used herein, a “compound of interest” refers to Compound 1 or a pharmaceutically acceptable salt thereof, or Compound 2 or a pharmaceutically acceptable salt thereof. The processes can further comprise milling the solidified melt, followed by compressing the milled product with one or more other excipients or ingredients (e.g., blending the milled product with one or more other excipients or ingredients and then compressing the blend mixture) to form a tablet, a mini-tablet, or a layer in a tablet. These other excipients or ingredients can include, for example, coloring agents, flavoring agents, lubricants or preservatives. Film-coating can also be added to the tablet or mini-tablet thus prepared.

In one embodiment, the melt is formed at a temperature of from 150 to 180° C. In another embodiment, the melt is formed at a temperature of from 150 to 170° C. In yet another embodiment, the melt is formed at a temperature of from 150 to 160° C. In yet another embodiment, the melt is formed at a temperature of from 160 to 170° C.

Any amorphous solid dispersion described or contemplated herein, including any amorphous solid dispersion described or contemplated in any aspect, embodiment, example, preference and composition of the invention, can be prepared according to any process described or contemplated herein.

In still another aspect, the present invention features solid pharmaceutical compositions prepared according to a process of the invention. Any process described or contemplated herein can be used to prepare a solid pharmaceutical composition comprising a compound of interest, a pharmaceutically acceptable hydrophilic polymer, and a pharmaceutically acceptable surfactant.

The present invention further features methods of using a solid pharmaceutical composition of the invention to treat HCV infection. The methods comprise administering a solid pharmaceutical composition of the invention to a patient in need thereof. The patient can be infected with HCV genotype 1, 2, 3, 4, 5 or 6.

The amorphous solid dispersion employed in the present invention can be prepared by a variety of techniques such as, without limitation, melt-extrusion, spray-drying, co-precipitation, freeze drying, or other solvent evaporation techniques, with melt-extrusion and spray-drying being preferred. The melt-extrusion process typically comprises the steps of preparing a melt which includes the active ingredient(s), the pharmaceutically acceptable hydrophilic polymer(s) and preferably the pharmaceutically acceptable surfactant(s), and then cooling the melt until it solidifies. “Melting” means a transition into a liquid or rubbery state in which it is possible for one component to get embedded, preferably homogeneously embedded, in the other component or components. In many cases, the polymer component(s) will melt and the other components including the active ingredient(s) and surfactant(s) will dissolve in the melt thereby forming a solution. Melting usually involves heating above the softening point of the polymer(s). The preparation of the melt can take place in a variety of ways. The mixing of the components can take place before, during or after the formation of the melt. For example, the components can be mixed first and then melted or be simultaneously mixed and melted. The melt can also be homogenized in order to disperse the active ingredient(s) efficiently. In addition, it may be convenient first to melt the polymer(s) and then to mix in and homogenize the active ingredient(s). In one example, all materials except surfactant(s) are blended and fed into an extruder, while the pharmaceutically acceptable surfactant(s) is molten externally and pumped in during extrusion.

To start a melt-extrusion process, the active ingredient(s) (e.g., Compound 1 or Compound 2) can be employed in their solid forms, such as their respective crystalline forms. The active ingredient(s) can also be employed as a solution or dispersion in a suitable liquid solvent such as alcohols, aliphatic hydrocarbons, esters or, in some cases, liquid carbon dioxide. The solvent can be removed, e.g. evaporated, upon preparation of the melt.

Various additives can also be included in the melt, for example, flow regulators (e.g., colloidal silica), binders, lubricants, fillers, disintegrants, plasticizers, colorants, or stabilizers (e.g., antioxidants, light stabilizers, radical scavengers, and stabilizers against microbial attack).

The melting and/or mixing can take place in an apparatus customary for this purpose. Particularly suitable ones are extruders or kneaders. Suitable extruders include single screw extruders, intermeshing screw extruders or multiscrew extruders, preferably twin screw extruders, which can be corotating or counterrotating and, optionally, be equipped with kneading disks. It will be appreciated that the working temperatures will be determined by the kind of extruder or the kind of configuration within the extruder that is used. Part of the energy needed to melt, mix and dissolve the components in the extruder can be provided by heating elements. However, the friction and shearing of the material in the extruder may also provide a substantial amount of energy to the mixture and aid in the formation of a homogeneous melt of the components.

The melt can range from thin to pasty to viscous. Shaping of the extrudate can be conveniently carried out by a calender with two counter-rotating rollers with mutually matching depressions on their surface. The extrudate can be cooled and allowed to solidify. The extrudate can also be cut into pieces, either before (hot-cut) or after solidification (cold-cut).

The solidified extrusion product can be further milled, ground or otherwise reduced to granules. The solidified extrudate, as well as each granule produced, comprises a solid dispersion, preferably a solid solution, of the active ingredient(s) in a matrix comprised of the pharmaceutically acceptable hydrophilic polymer(s) and the pharmaceutically acceptable surfactant(s). The extrusion product can also be blended with other active ingredient(s) and/or additive(s) before being milled or ground to granules. The granules can be further processed into suitable solid oral dosage forms.

In one example, copovidone and one or more surfactants (e.g., vitamin E TPGS) are mixed and granulated, followed by the addition of aerosil and a compound of interest. The mixture is milled, and then subject to extrusion. The extrudate thus produced can be milled and sieved for further processing to make capsules or tablets or mini-tablets. Surfactant(s) employed in this example can be added, for example, through liquid dosing during extrusion.

Preferably, in any aspect, embodiment, example, preference and composition of the invention where Compound 1 and Compound 2 are comprised in separate layers in a tablet, Compound 1 is melt-extruded at a temperature of from 155 to 180° C., and Compound 2 is melt-extruded at a temperature of from 150 to 195° C. For these cases, Compound 2 can also be melt-extruded at a temperature of from 150 to less than 222° C.

The generation of an acceptable amorphous Compound 2 extrudate has been found difficult. For instance, the particle size distribution (P SD) of the crystalline Compound 2 used for extrusion was shown to have a significant impact on extrudate appearance: the larger the particles the higher the risk to obtain a turbid extrudate with residual crystallinity Therefore, preferably, in any aspect, embodiment, example, preference and composition of the invention where Compound 1 and Compound 2 are comprised in separate layers in a tablet, before melt-extrusion, the crystalline Compound 2 is milled to particles with a median particle size (D50) of no more than 15 μm. More preferably, in any aspect, embodiment, example, preference and composition of the invention where Compound 1 and Compound 2 are comprised in separate layers in a tablet, before melt-extrusion, the crystalline Compound 2 is milled to particles with a median particle size (D50) of no more than 10 μm. Highly preferably, in any aspect, embodiment, example, preference and composition of the invention where Compound 1 and Compound 2 are comprised in separate layers in a tablet, before melt-extrusion, the crystalline Compound 2 is milled to particles with a median particle size of no more than 9 μm.

Also, preferably, in any aspect, embodiment, example, preference and composition of the invention where Compound 1 and Compound 2 are comprised in separate layers in a tablet, before melt-extrusion, the crystalline Compound 2 is milled to particles with a D90 of no more than 100 μm. More preferably, in any aspect, embodiment, example, preference and composition of the invention where Compound 1 and Compound 2 are comprised in separate layers in a tablet, before melt-extrusion, the crystalline Compound 2 is milled to particles with a D90 of no more than 80 μm. Highly preferably, in any aspect, embodiment, example, preference and composition of the invention where Compound 1 and Compound 2 are comprised in separate layers in a tablet, before melt-extrusion, the crystalline Compound 2 is milled to particles with a D90 of no more than 60 μm.

Preferably, in any aspect, embodiment, example, preference and composition of the invention where Compound 1 and Compound 2 are comprised in separate layers in a tablet, before melt-extrusion, the crystalline Compound 2 is milled to particles with a D50 of no more than 15 μm and a D90 of no more than 100 μm. More preferably, in any aspect, embodiment, example, preference and composition of the invention where Compound 1 and Compound 2 are comprised in separate layers in a tablet, before melt-extrusion, the crystalline Compound 2 is milled to particles with a D50 of no more than 10 μm and a D90 of no more than 80 μm. Highly preferably, in any aspect, embodiment, example, preference and composition of the invention where Compound 1 and Compound 2 are comprised in separate layers in a tablet, before melt-extrusion, the crystalline Compound 2 is milled to particles with a D50 of no more than 9 μm and a D90 of no more than 60 μm.

As used herein, particle size is measured by laser diffraction with Mastersizer. D90 refers to the particle size below which 90% of the particles exist.

The approach of solvent evaporation, via spray-drying, provides the advantage of allowing for processability at lower temperatures, if needed, and allows for other modifications to the process in order to further improve powder properties. The spray-dried powder can then be formulated further, if needed, and final drug product is flexible with regards to whether capsule, tablet, mini-tablet or any other solid dosage form is desired.

Exemplary spray-drying processes and spray-drying equipment are described in K. Masters, SPRAY DRYING HANDBOOK (Halstead Press, New York, 4^{th} ed., 1985). Non-limiting examples of spray-drying devices that are suitable for the present invention include spray dryers manufactured by Niro Inc. or GEA Process Engineering Inc., Buchi Labortechnik AG, and Spray Drying Systems, Inc. A spray-drying process generally involves breaking up a liquid mixture into small droplets and rapidly removing solvent from the droplets in a container (spray drying apparatus) where there is a strong driving force for evaporation of solvent from the droplets. Atomization techniques include, for example, two-fluid or pressure nozzles, or rotary atomizers. The strong driving force for solvent evaporation can be provided, for example, by maintaining the partial pressure of solvent in the spray drying apparatus well below the vapor pressure of the solvent at the temperatures of the drying droplets. This may be accomplished by either (1) maintaining the pressure in the spray drying apparatus at a partial vacuum; (2) mixing the liquid droplets with a warm drying gas (e.g., heated nitrogen); or (3) both.

The temperature and flow rate of the drying gas, as well as the spray dryer design, can be selected so that the droplets are dry enough by the time they reach the wall of the apparatus. This help to ensure that the dried droplets are essentially solid and can form a fine powder and do not stick to the apparatus wall. The spray-dried product can be collected by removing the material manually, pneumatically, mechanically or by other suitable means. The actual length of time to achieve the preferred level of dryness depends on the size of the droplets, the formulation, and spray dryer operation. Following the solidification, the solid powder may stay in the spray drying chamber for additional time (e.g., 5-60 seconds) to further evaporate solvent from the solid powder. The final solvent content in the solid dispersion as it exits the dryer is preferably at a sufficiently low level so as to improve the stability of the final product. For instance, the residual solvent content of the spray-dried powder can be less than 2% by weight. Highly preferably, the residual solvent content is within the limits set forth in the International Conference on Harmonization (ICH) Guidelines. In addition, it may be useful to subject the spray-dried composition to further drying to lower the residual solvent to even lower levels. Methods to further lower solvent levels include, but are not limited to, fluid bed drying, infra-red drying, tumble drying, vacuum drying, and combinations of these and other processes.

Like the solid extrudate described above, the spray dried product contains a solid dispersion, preferably a solid solution, of the active ingredient(s) in a matrix comprised of the pharmaceutically acceptable hydrophilic polymer(s) and the pharmaceutically acceptable surfactant(s).

Before feeding into a spray dryer, the active ingredient(s) (e.g., Compound 1 or Compound 2), the pharmaceutically acceptable hydrophilic polymer(s), as well as other excipients such as the pharmaceutically acceptable surfactant(s), can be dissolved in a solvent. Suitable solvents include, but are not limited to, alkanols (e.g., methanol, ethanol, 1-propanol, 2-propanol or mixtures thereof), acetone, acetone/water, alkanol/water mixtures (e.g., ethanol/water mixtures), or combinations thereof. The solution can also be preheated before being fed into the spray dryer.

The solid dispersion produced by melt-extrusion, spray-drying or other techniques can be prepared into any suitable solid oral dosage forms. In one embodiment, the solid dispersion prepared by melt-extrusion, spray-drying or other techniques (e.g., the extrudate or the spray-dried powder) can be compressed into tablets or mini-tablets. The solid dispersion can be either directly compressed, or milled or ground to granules or powders before compression. Compression can be done in a tablet press, such as in a steel die between two moving punches.

At least one additive selected from flow regulators, binders, lubricants, fillers, disintegrants, or plasticizers may be used in compressing the solid dispersion. These additives can be mixed with ground or milled solid dispersion before compacting. Disintegrants promote a rapid disintegration of the compact in the stomach and keeps the liberated granules separate from one another. Non-limiting examples of suitable disintegrants are cross-linked polymers such as cross-linked polyvinyl pyrrolidone, cross-linked sodium carboxymethylcellulose or sodium croscarmellose. Non-limiting examples of suitable fillers (also referred to as bulking agents) are lactose monohydrate, calcium hydrogenphosphate, microcrystalline cellulose (e.g., Avicell), silicates, in particular silicium dioxide, magnesium oxide, talc, potato or corn starch, isomalt, or polyvinyl alcohol. Non-limiting examples of suitable flow regulators include highly dispersed silica (e.g., colloidal silica such as Aerosil), and animal or vegetable fats or waxes. Non-limiting examples of suitable lubricants include polyethylene glycol (e.g., having a molecular weight of from 1000 to 6000), magnesium and calcium stearates, sodium stearyl fumarate, and the like.

Various other additives or ingredients may also be used in preparing a solid composition of the present invention, for example dyes such as azo dyes, organic or inorganic pigments such as aluminium oxide or titanium dioxide, or dyes of natural origin; stabilizers such as antioxidants, light stabilizers, radical scavengers, stabilizers against microbial attack; or other active pharmaceutical ingredients.

In order to facilitate the intake of a solid dosage form, it is advantageous to give the dosage form an appropriate shape. Large tablets that can be swallowed comfortably are therefore preferably elongated rather than round in shape.

A film coat on the tablet further contributes to the ease with which it can be swallowed. A film coat also improves taste and provides an elegant appearance. The film-coat usually includes a polymeric film-forming material such as polyvinyl alcohol, hydroxypropyl methylcellulose, hydroxypropylcellulose, and acrylate or methacrylate copolymers. Besides a film-forming polymer, the film-coat may further comprise a plasticizer, e.g. polyethylene glycol, a surfactant, e.g. polysorbates, and optionally a pigment, e.g. titanium dioxide or iron oxides. For instance, titanium dioxide can be used as an opacifier; and/or iron oxide red can be used as a colorant. The film-coating can also comprise a filler, e.g., lactose. The film-coating may also comprise talc as anti-adhesive. Preferably, the film coat accounts for less than 5% by weight of a pharmaceutical composition of the present invention. Higher amounts of the film coating can also be used.

All mini-tablets employed in the present invention can also be film coated. Preferably, the film coat accounts for no more than 30% by weight of each mini-tablet. More preferably, the film coat accounts for 10-20% by weight of each mini-tablet.

The present invention also unexpectedly found that in order for the mini-tablets described herein to provide adequate bioavailability similar to that of a regular tablet containing the same amount of drug in the same solid dispersion formulation, the mini-tablets need to be administered with food. Human clinical studies showed that food can significantly increase bioavailability of Compound 1 and Compound 2 formulated in mini-tablets and in solid dispersion form. For instance, without food, mini-tablets containing 200 mg of Compound 1 provided an AUC that was 41% lower than that provided by two regular tablets that contained the same amount of Compound 1 in the same solid dispersion formulation as in the mini-tablets. In comparison, when administered with food, the mini-tablets provided an AUC that was only 5% lower than that provided by the regular tablets. Likewise, when administered without food, mini-tablets containing 120 mg of Compound 2 provided an AUC that was 28% lower than that provided by three regular tablets that contained the same amount of Compound 2 in the same solid dispersion formulation as in the mini-tablets; however, when administered with food, the mini-tablets provided an AUC that was 6% higher than that provided by the regular tablets. All of the reference AUCs of the regular tablets were measured under fasting conditions.

Accordingly, the present invention features methods of treating HCV infection, wherein the methods comprise administering with food to a patient in need thereof a solid pharmaceutical composition of the invention that contains mini-tablets, such that the ratio of the Compound 1 AUC provided by the solid pharmaceutical composition over the Compound 1 AUC provided by a regular tablet comprising the same amount of Compound 1 in the same solid dispersion formulation as in the solid pharmaceutical composition is from 0.8 to 1.25, and the ratio of the Compound 2 AUC provided by the solid pharmaceutical composition over the Compound 2 AUC provided by a regular tablet comprising the same amount of Compound 2 in the same solid dispersion formulation as in the solid pharmaceutical composition is from 0.8 to 1.25. All AUCs are human AUCs, and all AUCs of the regular tablets are measured when the regular tablets are administered under fasting condition. Any composition described herein that contains mini-tablets can be used in these methods. The patient can be infected with HCV genotype 1, 2, 3, 4, 5 or 6.

In another aspect, the present invention features methods of treating HCV infection, wherein the methods comprise administering with food to a patient in need thereof a solid pharmaceutical composition of the invention that contains mini-tablets, such that the ratio of the Compound 1 AUC provided by the solid pharmaceutical composition over the Compound 1 AUC provided by a regular tablet comprising the same amount of Compound 1 (e.g., 100 mg) in the same solid dispersion formulation as in the solid pharmaceutical composition is from 0.8 to 1.25, and the ratio of the Compound 2 AUC provided by the solid pharmaceutical composition over the Compound 2 AUC provided by a regular tablet comprising the same amount of Compound 2 (e.g., 40 mg) in the same solid dispersion formulation as in the solid pharmaceutical composition is from 0.8 to 1.25. All AUCs are human AUCs, and all AUCs of the regular tablets are measured when the regular tablets are administered under fasting condition. Any composition described herein that contains mini-tablets can be used in these methods. The patient can be infected with HCV genotype 1, 2, 3, 4, 5 or 6.

In one aspect, this disclosure provides an oral dosage form comprising a powder, pellet, and/or granule (e.g., a film-coated granule) in a dispensing container. In certain embodiments, film-coated granules described herein are contained in a dispensing container. Examples of dispensing containers include tubes, packets or sachets, and individual wrappers. In some such embodiments, film-coated granules described herein are contained in a sachet. Such sachets are typically manufactured of paper, foil and/or plastic film.

In some embodiments, the oral dosage form includes film-coated granules containing Compound 1 and film-coated granules comprising Compound 2, wherein such film-coated granules are co-packaged in a dispensing container, preferably a sachet. In some embodiments, a sachet may contain one unit dose of the composition or a submultiple thereof, for example, about 319.0 mg of film-coated granules which contain about 50 mg of Compound 1 and/or about 242.4 mg of film-coated granules that contain about 20 mg of Compound 2.

In certain embodiments, an individual dispensing container (e.g., a sachet) includes about 40 mg of Compound 1 and about 20 mg of Compound 2, alternatively about 45 mg of Compound 1 and about 20 mg of Compound 2, alternatively about 50 mg of Compound 1 and about 20 mg of Compound 2, alternatively about 55 mg of Compound 1 and about 20 mg of Compound 2, alternatively about 40 mg of Compound 1 and about 15 mg of Compound 2, alternatively about 45 mg of Compound 1 and about 15 mg of Compound 2, alternatively about 50 mg of Compound 1 and about 15 mg of Compound 2, or alternatively about 55 mg of Compound 1 and about 15 mg of Compound 2.

In certain embodiments, an individual dispensing container (e.g., a sachet) includes a first amount of Compound 1 and a second amount of Compound 2, wherein the first and second amounts are each a submultiple of a desired dose of Compound 1 and Compound 2, respectively.

In some such embodiments, a sachet includes a first amount of Compound 1, wherein the first amount is a submultiple of a dose between 120 and 165 mg, alternatively between 130 and 165 mg, alternatively between 160 and 245 mg, alternatively between 180 and 220 mg, alternatively between 210 and 285 mg, or alternatively between 225 and 275 mg. In particular embodiments, the first amount is a submultiple of a dose of about 150, 200, and/or 250 mg. In one particular embodiment, the first amount is a submultiple of a dose of about 150, 200, and 200 mg (e.g., 5, 10, 25, or 50 mg).

In some such embodiments, a sachet includes a second amount of Compound 2, wherein the second amount is a submultiple of a dose between 45 and 75 mg, alternatively between 60 and 75 mg, alternatively between 60 and 90 mg, alternatively between 65 and 90 mg, alternatively between 75 and 110 mg, or alternatively between 85 and 110 mg. In particular embodiments, the second amount is a submultiple of a dose of about 60, 80, and/or 100 mg. In one particular embodiment, the second amount is a submultiple of a dose of about 60, 80, and 100 mg (e.g., 4, 5, 10, or 20 mg).

In another aspect, this disclosure provides methods for treating HCV infection, wherein the methods comprise administering to a patient in need thereof an oral dosage form comprising a first film-coated granule and a second film-coated granule, wherein the first film-coated granule contains Compound 1 and the second film-coated granule contains Compound 2.

In certain embodiments, the first film-coated granule and the second film-coated granule are co-packaged in a dispensing container such as a sachet.

In certain embodiments, the patient is a pediatric patient.

Any composition described herein that contains granules can be used in these methods. The patient can be infected with HCV genotype 1, 2, 3, 4, 5 or 6.

In yet another aspect, this disclosure provides methods for treating HCV infection in a pediatric patient, wherein the methods comprise administering Compound 1 and Compound 2 to the patient.

In certain embodiments, the pediatric patient is from 3 years old to less than 6 years old and Compound 1 is administered at a dose from about 120 to about 165 mg, preferably about 135 to about 165 mg. In certain embodiments, the pediatric patient is from 6 years old to less than 9 years old and Compound 1 is administered at a dose from about 160 to about 220 mg, preferably about 180 to about 220 mg. In certain embodiments, the pediatric patient is from 9 years old to less than 12 years old and Compound 1 is administered at a dose from about 210 to about 285 mg, preferably about 225 to about 275 mg.

In certain embodiments, the pediatric patient is from 3 years old to less than 6 years old and Compound 2 is administered at a dose from about 45 to about 75 mg. In certain embodiments, the pediatric patient is from 6 years old to less than 9 years old and Compound 2 is administered at a dose from about 60 to about 90 mg. In certain embodiments, the pediatric patient is from 9 years old to less than 12 years old and Compound 1 is administered at a dose from about 75 to about 110 mg.

In certain embodiments, (i) the pediatric patient is from 3 years old to less than 6 years old, Compound 1 is administered at a dose of about 150 mg, and Compound 2 is administered at a dose of about 60 mg; (ii) the patient is from 6 years old to less than 9 years old, Compound 1 is administered at a dose of about 200 mg, and Compound 2 is administered at a dose of about 80 mg; or (iii) the patient is from 9 years old to less than 12 years old, Compound 1 is administered at a dose of about 250 mg, and Compound 2 is administered at a dose of about 100 mg.

In certain embodiments, the pediatric patient is from 3 years old to less than 6 years old, Compound 1 is administered at a dose of about 150 mg, and Compound 2 is administered at a dose of about 60 mg. In certain embodiments, the pediatric patient is from 6 years old to less than 9 years old, Compound 1 is administered at a dose of about 200 mg, and Compound 2 is administered at a dose of about 80 mg. In certain embodiments, the pediatric patient is from 9 years old to less than 12 years old, Compound 1 is administered at a dose of about 250 mg, and Compound 2 is administered at a dose of about 100 mg.

In certain embodiments, the pediatric patient is from 3 years old to less than 6 years old, Compound 1 is administered at a dose of about 150 mg, and Compound 2 is administered at a dose of about 60 mg, and the patient obtains a sustained virologic response about 12 weeks post treatment (SVR12). In certain embodiments, the pediatric patient is from 6 years old to less than 9 years old, Compound 1 is administered at a dose of about 200 mg, and Compound 2 is administered at a dose of about 80 mg, and the patient obtains a sustained virologic response about 12 weeks post treatment (SVR12). In certain embodiments, the pediatric patient is from 9 years old to less than 12 years old, Compound 1 is administered at a dose of about 250 mg, and Compound 2 is administered at a dose of about 100 mg, and the patient obtains a sustained virologic response about 12 weeks post treatment (SVR12).

In certain embodiments, Compound 1 is administered from a first type of film-coated granule comprising an amorphous solid dispersion including (i) Compound 1, (ii) copovidone and (iii) Vitamin E TPGS. In some such embodiments, the total amount of Compound 1 comprised in the first type of granules is 50 mg.

In certain embodiments, Compound 2 is administered from a second type of film-coated granules comprising an amorphous solid dispersion including (i) Compound 2, (ii) copovidone and (iii) Vitamin E TPGS and propylene glycol monocaprylate. In some such embodiments, the total amount of Compound 2 comprised in the second type of granules is 20 mg.

Accordingly, the present invention features methods of treating HCV infection, wherein the methods comprise administering to a pediatric patient in need thereof a first type of film-coated granules containing Compound 1 and a second type of film-coated granules containing Compound 2 such that the ratio of the Compound 1 AUC provided by the first type of film-coated granules over the Compound 1 AUC provided by administration of a tablet comprising 100 mg of Compound 1 to an adult patient is from 0.8 to 1.25, and the ratio of the Compound 2 AUC provided by the second type of film-coated granules over the Compound 2 AUC provided by administration of a tablet comprising 40 mg of Compound 2 to an adult patient is from 0.8 to 1.25. All AUCs are human AUCs, and all AUCs of the regular tablets are measured when the regular tablets are administered under fasting condition.

In certain embodiments, the administration of the first type of film-coated granules results in a Compound 1 AUC that is bioequivalent to the Compound 1 AUC resulting from the administration of a tablet comprising 100 mg of Compound 1, and the administration of the second type of film-coated granules results in a Compound 2 AUC that is bioequivalent to the Compound 2 AUC resulting from the administration of a tablet comprising 40 mg of Compound 2. All AUCs are human AUCs, and all AUCs of the regular tablets are measured when the regular tablets are administered under fasting condition.

In certain embodiments, the Compound 1 AUC provided by the first type of film-coated granules is about 8670±268 ng·h/mL. In some embodiments, the Compound 1 AUC provided by the first type of film-coated granules is about 5970±179 ng·h/mL. In some embodiments, the Compound 1 AUC provided by the first type of film-coated granules is about 6700±244 ng·h/mL.

In certain embodiments, the Compound 1 AUC provided by the first type of film-coated granules is between about 8420 ng·h/mL and about 8938 ng·h/mL. In some embodiments, the Compound 1 AUC provided by the first type of film-coated granules is between about 5791 ng·h/mL and about 6149 ng·h/mL. In some embodiments, the Compound 1 AUC provided by the first type of film-coated granules is between about 6456 ng·h/mL and about 6944 ng·h/mL.

In certain embodiments, the Compound 1 AUC provided by the first type of film-coated granules is about 80% to about 125% of the Compound 1 geometric mean AUC. For example, in some embodiments, the Compound 1 AUC provided by the first type of film-coated granules is between about 6936 ng·h/mL and about 10838 ng·h/mL. In some embodiments, the Compound 1 AUC provided by the first type of film-coated granules is between about 4776 ng·h/mL and about 7463 ng·h/mL. In some embodiments, the Compound 1 AUC provided by the first type of film-coated granules is between about 5360 ng·h/mL and about 8375 ng·h/mL.

In certain embodiments, the Compound 2 AUC provided by the second type of film-coated granules is about 2300±114 ng·h/mL. In certain embodiments, the Compound 2 AUC provided by the second type of film-coated granules is about 1520±72 ng·h/mL. In certain embodiments, the Compound 2 AUC provided by the second type of film-coated granules is about 1660±59 ng·h/mL.

In certain embodiments, the Compound 2 AUC provided by the second type of film-coated granules is between about 2186 ng·h/mL and about 2414 ng·h/mL. In certain embodiments, the Compound 2 AUC provided by the second type of film-coated granules is between about 1448 ng·h/mL and about 1592 ng·h/mL. In certain embodiments, the Compound 2 AUC provided by the second type of film-coated granules is between about 1601 ng·h/mL and about 1719 ng·h/mL.

In certain embodiments, the Compound 2 AUC provided by the second type of film-coated granules is about 80% to about 125% of the Compound 2 geometric mean AUC. For example, in some embodiments, the Compound 2 AUC provided by the second type of film-coated granules is between about 1840 ng·h/mL and about 2875 ng·h/mL. In some embodiments, the Compound 2 AUC provided by the second type of film-coated granules is between about 1216 ng·h/mL and about 1900 ng·h/mL. In some embodiments, the Compound 2 AUC provided by the second type of film-coated granules is between about 1328 ng·h/mL and about 2075 ng·h/mL.

In certain embodiments, the first film-coated granules and the second film-coated granules are co-packaged in a dispensing container such as a sachet.

Any composition described herein that contains granules can be used in these methods. The patient can be infected with HCV genotype 1, 2, 3, 4, 5 or 6.

Various measures may be used to express the effectiveness of a method of the present invention. One such measure is SVR, which, as used herein, means that the virus is undetectable at the end of therapy and for at least 8 weeks after the end of therapy (SVR8); preferably, the virus is undetectable at the end of therapy and for at least 12 weeks after the end of therapy (SVR12); more preferably, the virus is undetectable at the end of therapy and for at least 16 weeks after the end of therapy (SVR16); and highly preferably, the virus is undetectable at the end of therapy and for at least 24 weeks after the end of therapy (SVR24). SVR24 is often considered as a functional definition of cure; and a high rate of SVR at less than 24 week post-treatment (e.g., SVR8 or SVR12) can be predictive of a high rate of SVR24.

Preferably, a method described herein achieves at least 70% SVR8. More preferably, a method described herein achieves at least 80% SVR8. Highly preferably, a method described herein achieves at least 90% SVR8. Most preferably, a method described herein achieves at least 95% SVR8. In certain embodiments, a patient treated with the method described herein obtains a sustained virologic response, post treatment at week 8 (SVR8).

Preferably, a method described herein achieves at least 70% SVR12. More preferably, a method described herein achieves at least 80% SVR12. Highly preferably, a method described herein achieves at least 90% SVR12. Most preferably, a method described herein achieves at least 95% SVR12. In certain embodiments, a patient treated with the method described herein obtains a sustained virologic response, post treatment, at week 12 (SVR12).

Preferably, a method described herein achieves at least 70% SVR16. More preferably, a method described herein achieves at least 80% SVR16. Highly preferably, a method described herein achieves at least 90% SVR16. In certain embodiments, a patient treated with the method described herein obtains a sustained virologic response, post treatment at week 16 (SVR16).

Preferably, a method described herein achieves at least 70% SVR24. More preferably, a method described herein achieves at least 80% SVR24. Highly preferably, a method described herein achieves at least 90% SVR24. In certain embodiments, a patient treated with the method described herein obtains a sustained virologic response, post treatment at week 24 (SVR24).

It should be understood that the above-described embodiments and the following examples are given by way of illustration, not limitation. Various changes and modifications within the scope of the present invention will become apparent to those skilled in the art from the present description.

### EXAMPLE 1

### Bilayer Film Coated Tablet

100 mg Compound 1 and 40 mg Compound 2 are prepared into a bilayer film-coated tablet. The composition of the bilayer film-coated tablet is shown in Table 1a or Table 1b. The tablet core consists of two layers, each based on an extrudate intermediate comprising Compound 1 (Table 2), and Compound 2 (Table 3), respectively. The compressed tablets are film-coated with a coating formulation based on hypromellose as non-functional coating.

TABLE 1a
Composition of Compound 1/Compound 2,
100 mg/40 mg Bilayer Film-Coated Tablet

| Ingredient | Amount (mg) |
|---|---|
| Compound 1, 20% extrusion granulation (see Table 2) | 500 |
| Compound 2, 10% extrusion granulation (see Table 3) | 400 |
| Croscarmellose sodium, Type Ac-Di-Sol ® | 26.3 |
| Colloidal silicon dioxide, Type Aerosil ® 200 | 4.7 |
| Sodium stearyl fumarate, Type Pruv ® | 4.7 |
| HPMC Coating | 37.4 |
| Total film-coated tablet | 973.1 |

TABLE 1b
Composition of Compound 1/Compound 2,
100 mg/40 mg Bilayer Film-Coated Tablet

| Ingredient | Amount (mg) |
|---|---|
| Compound 1, 20% extrusion granulation (see Table 2) | 500 |
| Compound 2, 10% extrusion granulation (see Table 3) | 400 |
| Croscarmellose sodium, Type Ac-Di-Sol ® | 26.3 |
| Colloidal silicon dioxide, Type Aerosil ® 200 | 4.7 |
| Sodium stearyl fumarate, Type Pruv ® | 4.7 |
| HPMC Coating | 28.1 |
| Total film-coated tablet | 963.8 |

TABLE 2
Composition of Compound 1, 20% Extrusion Granulation

| Ingredient | Amount (%, w/w) |
|---|---|
| Compound 1 | 20 |
| Copovidone, Type K 28 | 69 |
| Vitamin E Polyethylene Glycol Succinate | 10 |
| (Vitamin E TPGS) | |
| Colloidal silicon dioxide, Type Aerosil ® 200 | 1 |
| Total | 100 |

TABLE 3
Composition of Compound 2, 10% Extrusion Granulation

| Ingredient | Amount (%, w/w) |
|---|---|
| Compound 2 | 10. |
| Copovidon, Type K 28 | 79 |
| Vitamin E Polyethylene Glycol Succinate | 8 |
| (Vitamin E TPGS) | |
| Propylene Glycol Monocaprylate, Type II | 2 |
| (Capryol ™ 90) | |
| Colloidal silicon dioxide, Type Aerosil ® 200 | 1 |
| Total | 100 |

### EXAMPLE 2

### Mini-Tablets

Mini-tablets containing Compound 1 or Compound 2 can be prepared using the extrudates described in Tables 2 and 3 of Example 1, respectively. Manufacturing of Compound 1 mini-tablets can include the following steps: milling of the Compound 1 extrudate (e.g., the one described in Table 2 of Example 1), and then blending together with croscarmellose, colloidal silicon dioxide and sodium stearylfumarate, followed by tableting with a KORSCH XL 100 rotary press, using 19 fold 2 mm tableting tooling.

Manufacturing of Compound 2 mini-tablets can include the following steps: milling of the Compound 2 extrudate (e.g., the one described in Table 3 of Example 1), and then blending with colloidal silicon dioxide and sodium stearylfumarate, followed by tableting with a KORSCH XL 100 rotary press using, 19 fold 2 mm tableting tooling.

### EXAMPLE 3

### Film Coated Granules Contained in a Sachet

Granules comprising Compound 1 or Compound were prepared by blending extrudates with extra-granular excipients as generally described above in Examples 1 and 2. A milled extrudate blend comprising Compound 1 was compressed into granules (2 mm, diameter) and film-coated with a coating formulation based on hypromellose as non-functional coating. Similarly, a milled extrudate blend comprising Compound 2 was separately compressed into granules (2 mm, diameter) and separately film-coated with a coating formulation based on hypromellose as non-functional coating. The film-coated granules were then combined in a sachet.

The composition of a film-coated granule comprising Compound 1 is shown in Table 4.

TABLE 4
Composition of Compound 1, 15.7% Film Coated Granules

| | | Amount (mg)/ | Amount |
|---|---|---|---|
| | Ingredient | Unit (sachet) | (%, w/w) |
| Extrudate | Compound 1 | 50.0 | 15.7 |
| | Copovidone, Type K 28 | 172.5 | 54.1 |
| | Vitamin E Polyethylene | 25.0 | 7.8 |
| | Glycol Succinate (Vitamin | | |
| | E TPGS) | | |
| | Colloidal silicon dioxide | 2.5 | 0.8 |
| | 20% Compound 1 Extrudate | 250.0 | — |
| | Total Weight | | |
| Ext rag | Colloidal silicon dioxide | 1.35 | 0.4 |
| | Croscarmellose Sodium | 13.15 | 4.1 |
| | Sodium Stearyl Fumarate | 1.35 | 0.4 |
| | Unocated Core Granules | 265.85 | — |
| | Total Weight | | |
| Film Coat | HPMC Coating | 53.17 | 16.7 |
| | Film-Coated Granules | 319.02 | 100 |
| | Total Weight | | |

The composition of a film-coated granule comprising Compound 2 is shown in Table 5.

TABLE 5
Composition of Compound 2, 8.3% Film Coated Granules

| | | Amount (mg)/ | Amount |
|---|---|---|---|
| | Ingredient | Unit (sachet) | (%, w/w) |
| Extrudate | Compound 2 | 20.0 | 8.3 |
| | Copovidone, Type K 28 | 158.0 | 65.2 |
| | Vitamin E Polyethylene | 16.0 | 6.6 |
| | Glycol Succinate (Vitamin | | |
| | E TPGS) | | |
| | Propylene Glycol Mono- | 4.0 | 1.7 |
| | caprylate, Type II | | |
| | Colloidal silicon dioxide | 2.0 | 0.8 |
| | 10% Compound 2 Extrudate | 200.0 | — |
| | Total Weight | | |
| Extra- | Colloidal silicon dioxide | 1.0 | 0.4 |
| granular | Sodium Stearyl Fumarate | 1.0 | 0.4 |
| | Uncoated Core Granules | 202.0 | — |
| | Total Weight | | |
| Film Coat | HPMC Coating | 40.4 | 16.7 |
| | Film-Coated Granules | 242.4 | 100 |
| | Total Weight | | |

The film-coated granules of Table 4 were filled into a sachet with the film-coated granules of Table 5 to produce a 50 mg Compound 1/20 mg Compound 2 sachet.

### EXAMPLE 4

### Bioavailability and Effect of Food on Compound 1/Compound 2 Bilayer Tablets

Phase 1, single-dose, four-period, randomized, complete crossover clinical trials were conducted to determine the bioavailability and food effect of the Compound 1/Compound 2 film-coated bilayer tablets. Tablets described in Table 1b were used in Regimens A, B and C, and separate tablets containing either Compound 1 or Compound 2 were used in Regimen D.

Subjects took a single dose of Compound 1/Compound 2 on Day 1 of each Period. There was a washout of 4 days between doses.
i. Regimens A and D: study drugs were taken under fasting conditions.
ii. Regimen B: study drugs were taken approximately 30 minutes after start of moderate-fat breakfast (about 30% calories from fat).
iii. Regimen C: study drugs were taken approx. 30 minutes after start of high-fat breakfast (50% calories from fat).

The study design is summarized in Tables 6a and 6b. For Regimens A, B and C, the single dose consisted of three tablets of Table 1b, each tablet contains 100 mg/40 mg Compound 1/Compound 2. For Regimen D, the single dose contained three tablets of Compound 1, each of which contained 100 mg Compound 1, as well as three tablets of Compound 2, each of which contained 40 mg Compound 2.

TABLE 6a
Single Dose, Four-Period. Complete
Crossover Clinical Study Design

| Sequence | Number of | Regimens | | | |
|---|---|---|---|---|---|
| Number | Subjects | Period 1 | Period 2 | Period 3 | Period 4 |
| I | 6 | A | B | C | D |
| II | 6 | B | D | A | C |
| III | 6 | C | A | D | B |
| IV | 5 | D | C | B | A |

TABLE 6b
Single Dose, Four-Period, Complete
Crossover Clinical Study Design

| | |
|---|---|
| Regimen A | Single dose of Compound 1/Compound 2 film-coated |
| | bilayer tablets 300 mg/120 mg (3 × 100 mg/40 |
| | mg) given under fasting conditions |
| Regimen B | Single dose of Compound 1/Compound 2 film-coated |
| | bilayer tablets 300 mg/120 mg (3 × 100 mg/40 |
| | mg) given with a moderate fat breakfast |
| Regimen C | Single dose of Compound 1/Compound 2 film-coated |
| | bilayer tablets 300 mg/120 mg (3 × 100 mg/40 |
| | mg) given with a high fat breakfast |
| Regimen D | Single dose of Compound 1 tablets (300 mg, 3 × 100 |
| | mg tablets) and Compound 2 tablets (120 mg, 3 × 40 |
| | mg tablets) given under fasting conditions |

Table 7a shows the pharmacokinetic profiles of Compound 1 in these studies, as well as the food effect on the bioavailability of Compound 1. Table 7b shows the pharmacokinetic profiles of Compound 2, as well as the food effect on the bioavailability of Compound 2.

TABLE 7a
Compound 1 Pharmacokinetic Parameters ((Geometric Mean (Mean, CV %))

| Pharmacokinetic | | Regimen A | | Regimen B | | Regimen C | | Regimen D | |
|---|---|---|---|---|---|---|---|---|---|
| Parameters | Units | (N = 23) | | (N = 23) | | (N = 23) | | (N = 23) | |
| Cₘₐₓ | ng/mL | 294 | (384, 78) | 937 | (1193, 84) | 633 | (723, 54) | 803 | (973, 72) |
| Tₘₐₓ^{a} | h | 3.0 | (1.5 to 5.0) | 4.0 | (3.0 to 5.0) | 5.0 | (4.0 to 6.0) | 2.0 | (1.0 to 3.0) |
| t_{1/2}^{b} | h | 6.0 | (24) | 6.0 | (16) | 6.3 | (18) | 5.7 | (16) |
| AUCₜ | ng · h/mL | 1150 | (1430, 70) | 3040 | (3460, 60) | 2110 | (2390, 54) | 2620 | (2970, 53) |
| AUC_{inf} | ng · h/mL | 1150 | (1440, 69) | 3040 | (3470, 60) | 2120 | (2390, 54) | 2620 | (2980, 53) |

^{a}Median (Minimum to Maximum)
^{b}Harmonic mean (pseudo % CV)

TABLE 7b
Compound 2 Pharmacokinetic Parameters ((Geometric Mean (Mean, CV %))

| Pharmacokinetic | | Regimen A | | Regimen B | | Regimen C | | Regimen D | |
|---|---|---|---|---|---|---|---|---|---|
| Parameters | Units | (N = 23) | | (N = 23) | | (N = 23) | | (N = 23) | |
| Cₘₐₓ | ng/mL | 116 | (140, 60) | 221 | (239, 44) | 237 | (262, 45) | 175 | (192, 38) |
| Tₘₐₓ^{a} | h | 4.0 | (2.0 to 5.0) | 5.0 | (3.0 to 5.0) | 5.0 | (4.0 to 6.0) | 4.0 | (2.0 to 5.0) |
| t_{1/2}^{b} | h | 13.3 | (9) | 13.0 | (10) | 13.5 | (9) | 12.5 | (8) |
| AUCₜ | ng · h/mL | 910 | (1100, 64) | 1280 | (1400, 49) | 1390 | (1560, 49) | 1420 | (1570, 40) |
| AUC_{inf} | ng · h/mL | 960 | (1160, 64) | 1350 | (1480, 49) | 1460 | (1650, 50) | 1490 | (1650, 40) |

^{a}Median (Minimum to Maximum)
^{b}Harmonic mean (pseudo % CV)

The above studies showed that administration with food significantly improved the bioavailability of both Compound 1 and Compound 2, and the improvement was achieved with regard to the fat content in the food. Additional studies comparing film-coated to uncoated bilayer tablets further showed that film-coating had minimal impact on the bioavailability of co-formulated Compound 1 and Compound 2.

### EXAMPLE 5

### Bioavailability of Compound 1/Compound 2 Mini-Tablets

14 subjects were enrolled in this study and dosed with co-formulated Compound 1/Compound 2 in mini-tablets. The study design is summarized in Tables 8a and 8b. One subject spilled 4 mini-tablets (out of 100-150 total mini-tablets) during dosing of Period 2 (Regimen G) and was not excluded from the analysis. The mini-tablets were prepared according to a process similar to that described in Example 2.

TABLE 8a
Single Dose, Crossover Clinical Study Design

| Sequence | Number of | Regimens | | |
|---|---|---|---|---|
| Number | Subjects | Period 1 | Period 2 | Period 3 |
| VII | 5 | F | G | J |
| VIII | 5 | G | J | F |
| IX | 5 | J | F | G |

TABLE 8b
Single Dose, Crossover Clinical Study Design

| | |
|---|---|
| Regimen F | Single dose of Compound 1/Compound 2 mini-tablets |
| | given under fasting conditions (total dose of 200 |
| | mg/120 mg Compound 1/Compound 2) |
| Regimen G | Single dose of Compound 1/Compound 2 mini-tablets |
| | given under non-fasting conditions (total dose of |
| | 200 mg/120 mg Compound 1/Compound 2) |
| Regimen J | Single dose of two Compound 1 tablets (each containing |
| | 100 mg Compound 1) and three Compound 2 tablets |
| | (each containing 40 mg Compound 2) under fasting |
| | conditions |

Table 9a shows the pharmacokinetic profiles of Compound 1 in these studies, as well as the food effect on the bioavailability of Compound 1. Table 9b shows the pharmacokinetic profiles of Compound 2, as well as the food effect on the bioavailability of Compound 2.

TABLE 9a
Compound 1 Pharmacokinetic Parameters ((Geometric Mean (Mean, CV %))

| Pharmacokinetic | | Regimen F | | Regimen G | | Regimen J | |
|---|---|---|---|---|---|---|---|
| Parameters | Units | (N = 14) | | (N = 14) | | (N = 14) | |
| Cₘₐₓ | ng/mL | 123 | (164, 103) | 166 | (314, 209) | 212 | (333, 159) |
| Tₘₐₓ^{a} | h | 1.0 | (0.5 to 4.0) | 1.75 | (1.0 to 4.0) | 1.5 | (0.5 to 3.0) |
| t_{1/2}^{b} | h | 5.61 | (29) | 6.42 | (31) | 5.93 | (39) |
| AUCₜ | ng · h/mL | 428 | (598, 107) | 699 | (1020, 150) | 738 | (1150, 165) |
| AUC_{inf} | ng · h/mL | 432 | (602, 107) | 704 | (1020, 149) | 742 | (1160, 164) |

^{a}Median (Minimum to Maximum)
^{b}Harmonic mean (pseudo % CV)

TABLE 9b
Compound 2 Pharmacokinetic Parameters ((Geometric Mean (Mean, CV %))

| Pharmacokinetic | | Regimen F | | Regimen G | | Regimen J | |
|---|---|---|---|---|---|---|---|
| Parameters | Units | (N = 14) | | (N = 14) | | (N = 14) | |
| Cₘₐₓ | ng/mL | 96.0 | (110, 61) | 177 | (198, 55) | 139 | (169, 75) |
| Tₘₐₓ^{a} | h | 4.0 | (2.0 to 6.0) | 3.0 | (3.0 to 5.0) | 4.5 | (1.5 to 6.0) |
| t_{1/2}^{b} | h | 13.4 | (15) | 13.2 | (10) | 13.3 | (7) |
| AUCₜ | ng · h/mL | 863 | (1050, 80) | 1250 | (1480, 70) | 1190 | (1570,91) |
| AUC_{inf} | ng · h/mL | 913 | (1110, 80) | 1320 | (1560,71) | 1260 | (1660, 92) |

^{a}Median (Minimum to Maximum)
^{b}Harmonic mean (pseudo % CV)

The above studies showed that administration with food significantly increased the bioavailability of both Compound 1 and Compound 2 when delivered in co-formulated mini-tablets.

### EXAMPLE 6

### Bioavailability of Compound 1/Compound 2 Film-Coated Granules

The study was designed to assess the steady state AUC and to assess the pharmacokinetics (PK) of Compound 1/Compound 2 in pediatric subjects by age group. Surprisingly, this study was not straightforward due the drug-drug interaction between Compound 1 and Compound 2, the non-linear pharmacokinetic profile of Compound 1 and Compound 2 in the studied age groups, as well as other unpredictable variables.

Subjects were enrolled in this study and dosed with film-coated granules containing Compound 1/Compound 2 as generally described above in Example 3. The study groups are summarized in Table 10.

TABLE 10
Dosing of First and Second Sets of Subjects

| | | Dosage (Compound 1/Compound 2) | |
|---|---|---|---|
| | | Pre-Adjustment | Post-Adjustment |
| | Number of | (40 mg/15 mg | (50 mg/20 mg |
| Group | Sachets | per sachet) | per sachet) |
| 9 to <12 years old | 5 | 200 mg/75 mg | 250 mg/100 mg |
| 6 to <9 years old | 4 | 160 mg/60 mg | 200 mg/80 mg |
| 3 to <6 years old | 3 | 120 mg/45 mg | 150 mg/60 mg |

An intensive PK sample draw was done at the Week 2 visit with blood samples taken immediately prior to dose (0 hour) and at 2, 4, 6, and 12 hours post dose.

Pre-Adjustment Groups.

Table 11a shows the pharmacokinetic profiles of Compound 1 in the pre-adjustment groups. Table 11b shows the pharmacokinetic profiles of Compound 2 in the pre-adjustment groups.

Target AUC was derived from the geometric mean of AUC values from a population of adult subjects who received Compound 1 and Compound 2. Target AUC for Compound 1 was determined to be 4800 hr*ng/mL. Target AUC for Compound 2 was determined to be 1430 hr*ng/mL.

TABLE 11a
Compound 1 AUC Values at Week 2 in Pre-Adjustment Subjects

| | | |
|---|---|---|
| Group | Geometric Mean (% CV) | Target AUC/AUC |

| | | | |
|---|---|---|---|
| 9 to <12 years old | 4070 | (211) | 1.18 |
| 6 to <9 years old | 1680 | (94) | 2.86 |
| 3 to <6 years old | 3030 | (52) | 1.58 |

TABLE 11b
Compound 2 AUC Values at Week 2 in Pre-Adjustment Subjects

| | | |
|---|---|---|
| Group | Geometric Mean (% CV) | Target AUC/AUC |

| | | | |
|---|---|---|---|
| 9 to <12 years old | 1250 | (79) | 1.14 |
| 6 to <9 years old | 987 | (87) | 1.44 |
| 3 to <6 years old | 871 | (63) | 1.64 |

As shown in Tables 11a and 11b, the average exposures in each of the cohorts were lower than the targeted AUC.

Post-Adjustment Groups.

The lower than expected AUC values observed with the pre-adjustment dose (40 mg Compound 1+15 mg Compound per sachet) led to a dose adjustment in subsequent subjects.

Table 12a shows the pharmacokinetic profiles of Compound 1 in the post-adjustment groups. Table 12b shows the pharmacokinetic profiles of Compound 2 in the post-adjustment groups.

TABLE 12a
Compound 1 AUC and C_{trough} Values at
Week 2 in Post-Adjustment Subjects

| Group | N | AUC | C_{trough} | |
|---|---|---|---|---|
| 9 to <12 years old | 10 | 8670 (268) | 17.0 | (1310) |
| 6 to <9 years old | 9 | 5970 (179) | 8.2 | (576) |
| 3 to <6 years old | 10 | 6700 (244) | 9.8 | (343) |

TABLE 12b
Compound 2 AUC and C_{trough} Values at
Week 2 in Post-Adjustment Subjects

| Group | N | AUC | | C_{trough} |
|---|---|---|---|---|
| 9 to <12 years old | 10 | 2300 | (114) | 40.8 (194) |
| 6 to <9 years old | 9 | 1520 | (72) | 20.0 (119) |
| 3 to <6 years old | 10 | 1660 | (59) | 17.5 (139) |

The predicted PK exposures (“Pop-PK AUC”) of Compound 1/Compound 2 based on all PK information collected in the study (including data from other study weeks) is shown in Table 13 and Table 13a.

TABLE 13
Compound 1/Compound 2 AUC/Target AUC Ratio

| | | Compound 1 | | Compound 2 | |
|---|---|---|---|---|---|
| | | Pop- | AUC/ | Pop- | AUC/ |
| Group | N | PK AUC | AUC_{Targ} | PK AUC | AUC_{Targ} |
| 9 to <12 years old | 10 | 5180 | 1.08 | 1440 | 1.00 |
| 6 to <9 years old | 9 | 5770 | 1.20 | 1460 | 1.02 |
| 3 to <6 years old | 10 | 6310 | 1.31 | 1430 | 1.00 |

Across the 3 cohorts of pediatric subjects , the AUC/AUC_{Targ} geometric mean ratio is from 1.08 to 1.31 for Compound 1 and from 1.00 to 1.02 for Compound 2.

TABLE 13a
Compound 1/Compound 2 AUC/Target AUC Ratio

| | | Compound 1 | | Compound 2 | |
|---|---|---|---|---|---|
| | | Pop- | AUC/ | Pop- | AUC/ |
| Group | N | PK AUC | AUC_{Targ} | PK AUC | AUC_{Targ} |
| 9 to <12 years old | 10 | 8120 | 1.7 | 2364 | 1.64 |
| 6 to <9 years old | 9 | 5160 | 1.07 | 1733 | 1.20 |
| 3 to <6 years old | 10 | 8990 | 1.87 | 1757 | 1.22 |

Across the 3 cohorts of pediatric subjects , the AUC/AUC_{Targ} geometric mean ratio is from 1.07 to 1.87 for Compound 1 and from 1.22 to 1.64 for Compound 2.

### EXAMPLE 7

### Treatment of Pediatric Patients

Children aged 3 to less than 12 years and weighing 12 kg to less than 45 kg

The recommended treatment durations for HCV genotype 1, 2, 3, 4, 5, or 6 infected patients with compensated liver disease (with or without cirrhosis) are provided in Table 14 and Table 15. The number of sachets and dosage based on body weight for children are shown in Table 16. The sachets should be taken together, with food once daily.

TABLE 14
Recommended treatment duration for
patients without prior HCV therapy

Recommended treatment duration

| Genotype | No cirrhosis | Cirrhosis | |
|---|---|---|---|
| GT 1, 2, 4, 5, 6 | 8 weeks | 8 | weeks |
| GT 3 | 8 weeks | 12 | weeks |

TABLE 15
Recommended treatment duration for patients who
failed prior therapy with peg-IFN + ribavirin
+/− sofosbuvir, or sofosbuvir + ribavirin

| | Recommended treatment duration | | |
|---|---|---|---|
| Genotype | | No cirrhosis | Cirrhosis |

| | | | |
|---|---|---|---|
| GT 1, 2, 4-6 | 8 | weeks | 12 weeks |
| GT 3 | 16 | weeks | 16 weeks |

TABLE 16
Recommended dosage for children 3 to <12 years of age

| Weight of child | Number of sachets once daily | |
|---|---|---|
| (kg) | (Compound 1 + Compound 2) | |
| ≥12 to <20 kg | 3 sachets | (150 mg + 60 mg) |
| ≥20 to <30 kg | 4 sachets | (200 mg + 80 mg) |
| ≥30 to <45 kg | 5 sachets | (250 mg + 100 mg) |

The adult dose of Compound 1/Compound 2 tablets should be used in children weighing 45 kg or greater.

The coated granule formulation is intended for children 3 to less than 12 years or weighing 12 kg to less than 45 kg. Children weighing 45 kg or more should use the tablet formulation. Because the formulations have different pharmacokinetic profiles, the tablets and the coated granules are not interchangeable.

Method of Administration

Coated Granules in Sachet for Oral Administration

Patients should be instructed to take the recommended dose with food once daily.

In addition, the granules for the total daily dose should be sprinkled on a small amount of soft food with a low water content that will stick to a spoon and can be swallowed without chewing (e.g., peanut butter, chocolate hazelnut spread, soft/cream cheese, thick jam, or Greek yogurt).

Liquids or foods that would drip or slide off the spoon should not be used as the medication may dissolve quickly and become less effective.

The mixture of food and granules should be swallowed immediately; the granules should not be crushed or chewed.

Clinical Efficacy and Safety

DORA (Part 2) was an open-label study to evaluate safety and efficacy in 48 children aged 3 years to less than 12 years who received weight-based coated granules in sachet for oral administration for 8 weeks. Eighteen subjects received the initial lower dose, and 30 subjects received the final recommended dose. The median age was 7 years (range: 3 to 11); 75% had HCV genotype 1; 23% had HCV genotype 3; 2% had HCV genotype 4; 60% were female; 6% were Black; all were HCV treatment-naïve; none had cirrhosis; the mean weight was 26 kg (range: 13 to 44). In subjects receiving the recommended dose, the SVR12 rate was 100% (30/30). No subject taking the recommended dose experienced virologic failure.

At the recommended doses according to the patient's body weight, exposures of Compound 1 and Compound 2 in children aged 3 to <12 years were comparable to those in adolescents aged 12 years to <18 years and in adults from Phase 2/3 studies.

Pharmacokinetic Properties

The pharmacokinetic properties of Compound 1/Compound 2 are provided in Table 17.

TABLE 17
Pharmacokinetic properties of the components
of Compound 1/Compound 2 in healthy subjects

| | |
|---|---|
| Compound 1 | Compound 2 |

Absorption

| | | |
|---|---|---|
| Tₘₐₓ (h)^{a} of tablets | 5.0 | 5.0 |
| Tₘₐₓ (h)^{a} of granules | 3.0-4.0 | 3.0-5.0 |
| Effect of meal (relative to | ↑ 83-163% | ↑ 40-53% |
| fasting)^{b} on adult tablets | | |
| Effect of meal (relative to | ↑ 131-168% | ↑ 56-115% |
| fasting)b on granules | | |

Distribution

| | | |
|---|---|---|
| % Bound to human plasma | 97.5 | >99.9 |
| proteins | | |
| Blood-to-plasma ratio | 0.57 | 0.62 |

Biotransformation

| | | |
|---|---|---|
| Metabolism | Secondary | none |

Elimination

| | | |
|---|---|---|
| Major route of elimination | Biliary | Biliary |
| | excretion | excretion |
| t_{1/2} (h) at steady-state | 6-9 | 23-29 |
| % of dose excreted in urine^{c} | 0.7 | 0 |
| % of dose excreted in faeces^{c} | 92.1^{d} | 96.6 |

Transport

| | | |
|---|---|---|
| Substrate of transporter | P-gp, | P-gp and |
| | BCRP, and | not excluded |
| | OATP1B1/3 | BCRP |

^{a}Median Tₘₐₓ following single doses of Compound 1 and Compound 2 in healthy subjects.
^{b}Mean systemic exposure with moderate to high fat meals.
^{c}Single dose administration of [¹⁴C] Compound 1 or [¹⁴C] Compound 2 in mass balance studies.
^{d}Oxidative metabolites or their byproducts accounted for 26% of radioactive dose. No Compound 1 metabolites were observed in plasma.

The foregoing description of the present invention provides illustration and description, but is not intended to be exhaustive or to limit the invention to the precise one disclosed. Modifications and variations are possible in light of the above teachings or may be acquired from practice of the invention. Thus, it is noted that the scope of the invention is defined by the claims and their equivalents.

1. A method for treating hepatitis C virus (HCV) infection in a pediatric patient, comprising administering a film-coated granule composition comprising
50 mg of Compound 1 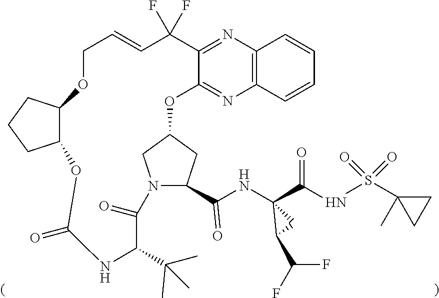 [SMILES: CC(C)(C)[C@@H]1NC(=O)O[C@@H]2CCC[C@H]2OC/C=C/C(F)(F)c2nc3ccccc3nc2O[C@@H]2C[C@@H](C(=O)N[C@]3(C(=O)NS(=O)(=O)C4(C)CC4)C[C@H]3C(F)F)N(C2)C1=O]
and
20 mg of Compound 2 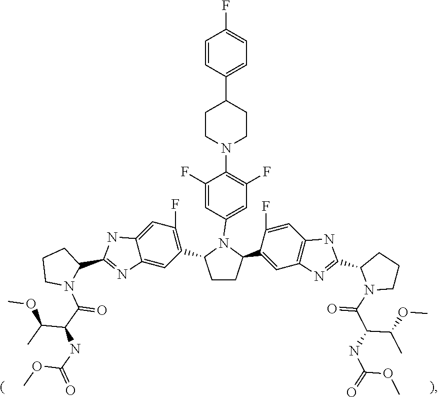
wherein the film-coated granule composition is provided in a sachet, and
wherein
the patient is from 3 years old to less than 6 years old and three sachets are administered, comprising a total of about 150 mg of Compound 1, and about 60 mg of Compound 2, and the patient obtains a sustained virologic response about 12 weeks post treatment (SVR12).

2-4. (canceled)

5. The method of claim 1, wherein Compound 1 is present in a first type of film-coated granule comprising an amorphous solid dispersion including (i) Compound 1, (ii) copovidone and (iii) Vitamin E TPGS.

6. The method of claim 5, wherein the total amount of Compound 1 comprised in the first type of granule is 50 mg.

7. The method of claim 1, wherein Compound 2 is present in a second type of film-coated granule comprising an amorphous solid dispersion including (i) Compound 2, (ii) copovidone and (iii) Vitamin E TPGS and propylene glycol monocaprylate.

8. The method of claim 7, wherein the total amount of Compound 2 comprised in the second type of granule is 20 mg.

9. A stable, oral, immediate release solid pharmaceutical composition comprising:
(1) 50 mg of Compound 1 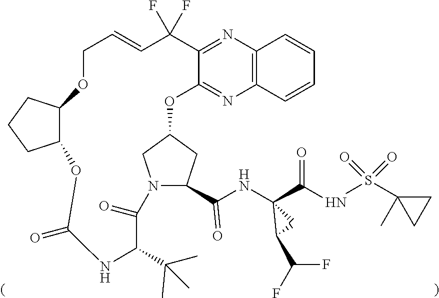 [SMILES: CC(C)(C)[C@@H]1NC(=O)O[C@@H]2CCC[C@H]2OC/C=C/C(F)(F)c2nc3ccccc3nc2O[C@@H]2C[C@@H](C(=O)N[C@]3(C(=O)NS(=O)(=O)C4(C)CC4)C[C@H]3C(F)F)N(C2)C1=O]
formulated in an amorphous solid dispersion which further comprises from 50% to 80% by weight of a first pharmaceutically acceptable polymer and from 5% to 15% by weight of a first pharmaceutically acceptable surfactant; and
(2) 20 mg of Compound 2 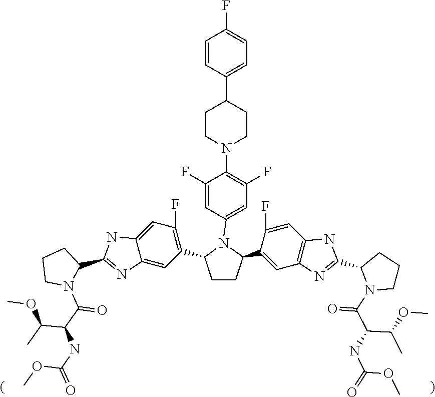
formulated in an amorphous solid dispersion which further comprises from 50% to 90% by weight of a second pharmaceutically acceptable polymer and from 5% to 15% by weight of a second pharmaceutically acceptable surfactant,
wherein the composition is provided in a sachet and is stable for the duration of a shelf life of about 24 months in the sachet.

10. The solid pharmaceutical composition of claim 9, where the composition is a mixture of (1) a first type of film-coated granule including the 50 mg of Compound 1 and (2) a second type of film-coated granule including the 20 mg of Compound 2.

11. The solid pharmaceutical composition of claim 9, wherein the amorphous solid dispersion in which Compound 1 is formulated comprises 20% by weight of Compound 1, and the amorphous solid dispersion in which Compound 2 is formulated comprises 10% by weight of Compound 2.

12. The solid pharmaceutical composition of claim 11, where the composition is a mixture of (1) a first type of film-coated granule including the 50 mg of Compound 1 and (2) a second type of film-coated granule including the 20 mg of Compound 2.

13. The solid pharmaceutical of claim 12, wherein the first and second polymers are copovidone, and the first and second surfactants are Vitamin E TPGS.

14. The solid pharmaceutical composition of claim 12, wherein the first and second polymers are copovidone, and the first surfactant is Vitamin E TPGS, and the second surfactant is a combination of Vitamin E TPGS and propylene glycol monocaprylate.

15. The solid pharmaceutical composition of claim 9 wherein the composition has an in vitro release profile according to at least one of the following profiles:
(i) when the composition is dissolved in 500 mL of a dissolution medium using a standard USP dissolution Apparatus 1 (basket) operating at 75 RPM at 37° C., at least 80% of Compound 1 in the composition is released within 40 minutes and at least 80% of Compound 2 in the composition is released within 40 minutes, wherein the dissolution medium is 0.1 M Acetate buffer (pH 4.0) with 1% Polysorbate 80;
(ii) when the composition is dissolved in 500 mL of a dissolution medium using a standard USP dissolution Apparatus 1 (basket) operating at 75 RPM at 37° C., at least 30% of Compound 1 in the composition is released within 20 minutes and at least 45% of Compound 2 in the composition is released within 20 minutes, wherein the dissolution medium is 0.1 M Acetate buffer (pH 4.0) with 1% Polysorbate 80; or
(iii) when the composition is dissolved in 500 mL of a dissolution medium using a standard USP dissolution Apparatus 1 (basket) operating at 75 RPM at 37° C., at least 5% of Compound 1 in the composition is released within 10 minutes and at least 10% of Compound 2 in the composition is released within 10 minutes, wherein the dissolution medium is 0.1 M Acetate buffer (pH 4.0) with 1% Polysorbate 80.

16. The solid pharmaceutical composition of claim 9, wherein a single dose of three sachets administered to a population of healthy, non-fasted patients from 3 years old to less than 6 years old results in a mean AUC value between about 6936 ng·h/mL and about 10838 ng·h/mL for Compound 1, and a mean AUC value between about 1840 ng·h/mL and about 2875 ng·h/mL for Compound 2.

17. The solid pharmaceutical composition of claim 9, wherein a single dose of four sachets administered to a population of healthy, non-fasted patients from 6 years old to less than 9 years old results in a mean AUC value between about 4776 ng·h/mL and about 7463 ng·h/mL for Compound 1, and a mean AUC value between about 1216 ng·h/mL and about 1900 ng·h/mL for Compound 2.

18. The solid pharmaceutical composition of claim 9, wherein a single dose of five sachets administered to a population of healthy, non-fasted patients from 9 years old to less than 12 years old results in a mean AUC value between about 5360 ng·h/mL and about 8375 ng·h/mL for Compound 1, and a mean AUC value between about 1328 ng·h/mL and about 2075 ng·h/mL for Compound 2.

19. A pharmaceutical composition that is bioequivalent to a solid pharmaceutical composition comprising:
(1) a first type of film-coated granule, comprising:
a. 250 mg of a 20% Compound 1 extrusion granulation, comprising:
i. 50 mg of Compound 1 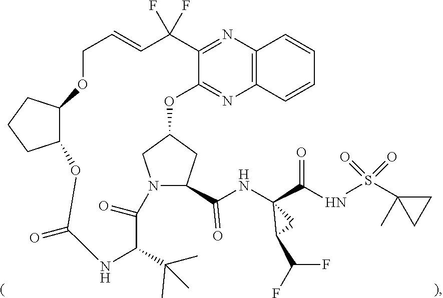 [SMILES: CC(C)(C)[C@@H]1NC(=O)O[C@@H]2CCC[C@H]2OC/C=C/C(F)(F)c2nc3ccccc3nc2O[C@@H]2C[C@@H](C(=O)N[C@]3(C(=O)NS(=O)(=O)C4(C)CC4)C[C@H]3C(F)F)N(C2)C1=O]
ii. 172.5 mg copovidone,
iii. 25.0 vitamin E TPGS, and
iv. 2.5 mg colloidal silicon dioxide;
b. 1.35 mg colloidal silicon dioxide;
c. 13.15 mg croscarmellose sodium;
d. 1.35 mg sodium stearyl fumarate; and
e. 53.17 mg HPMC coating; and
(2) a second type of film-coated granule, comprising:
a. 200 mg of a 10% Compound 2 extrusion granulation, comprising:
i. 20 mg of Compound 2 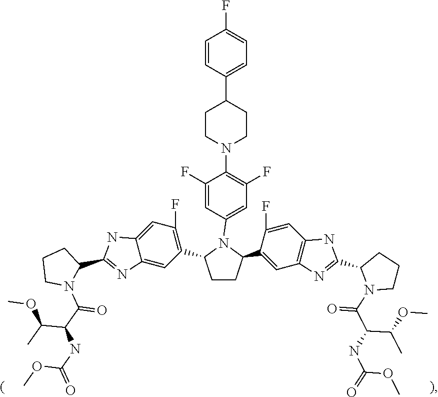
ii. 158.2 mg copovidone,
iii. 16.0 mg vitamin E TPGS,
iv. 4.0 mg propylene glycol monocaprylate, and
v. 2.0 mg colloidal silicone dioxide;
c. 1.0 mg colloidal silicon dioxide;
d. 1.0 mg sodium stearyl fumarate; and
e. 40.4 mg HPMC coating.

20. A method for treating hepatitis C virus (HCV) infection, comprising administering a pharmaceutical composition of claim 9 to a patient in need thereof, wherein the patient obtains a sustained virologic response about 12 weeks post treatment (SVR12).

21. A method for treating hepatitis C virus (HCV) infection in a pediatric patient, comprising administering a film-coated granule composition comprising
50 mg of Compound 1 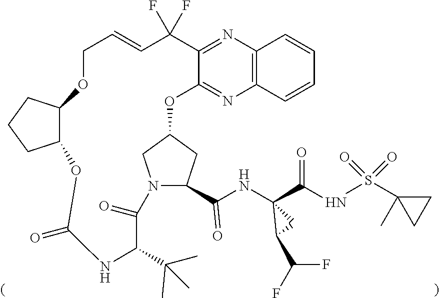 [SMILES: CC(C)(C)[C@@H]1NC(=O)O[C@@H]2CCC[C@H]2OC/C=C/C(F)(F)c2nc3ccccc3nc2O[C@@H]2C[C@@H](C(=O)N[C@]3(C(=O)NS(=O)(=O)C4(C)CC4)C[C@H]3C(F)F)N(C2)C1=O]
and
20 mg of Compound 2 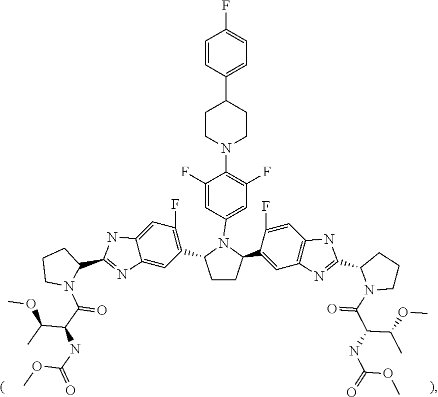
wherein the film-coated granule composition is provided in a sachet, and
wherein the patient is from 6 years old to less than 9 years old and four sachets are administered, comprising a total of about 200 mg of Compound 1, and about 80 mg of Compound 2, and the patient obtains a sustained virologic response about 12 weeks post treatment (SVR12).

22. The method of claim 21, wherein Compound 1 is present in a first type of film-coated granule comprising an amorphous solid dispersion including (i) Compound 1, (ii) copovidone and (iii) Vitamin E TPGS.

23. The method of claim 22, wherein the total amount of Compound 1 comprised in the first type of granule is 50 mg.

24. The method of claim 21, wherein Compound 2 is present in a second type of film-coated granule comprising an amorphous solid dispersion including (i) Compound 2, (ii) copovidone and (iii) Vitamin E TPGS and propylene glycol monocaprylate.

25. The method of claim 24, wherein the total amount of Compound 2 comprised in the second type of granule is 20 mg.

26. A method for treating hepatitis C virus (HCV) infection in a pediatric patient, comprising administering a film-coated granule composition comprising
50 mg of Compound 1 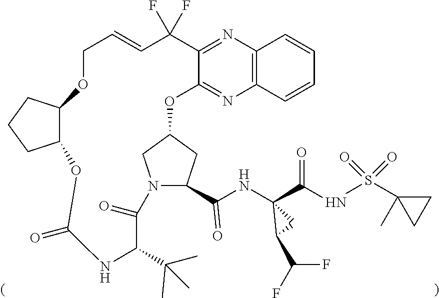 [SMILES: CC(C)(C)[C@@H]1NC(=O)O[C@@H]2CCC[C@H]2OC/C=C/C(F)(F)c2nc3ccccc3nc2O[C@@H]2C[C@@H](C(=O)N[C@]3(C(=O)NS(=O)(=O)C4(C)CC4)C[C@H]3C(F)F)N(C2)C1=O]
and
20 mg of Compound 2 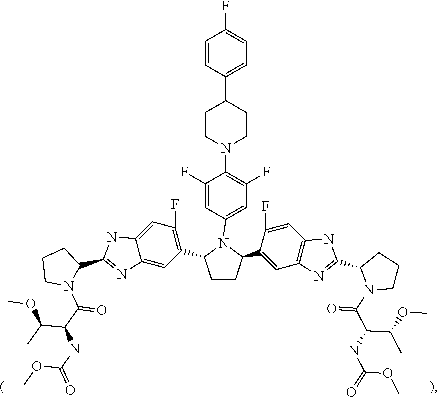
wherein the film-coated granule composition is provided in a sachet, and
wherein the patient is from 9 years old to less than 12 years old and five sachets are administered, comprising a total of about 250 mg of Compound 1, and about 100 mg of Compound 2, and the patient obtains a sustained virologic response about 12 weeks post treatment (SVR12).

27. The method of claim 26, wherein Compound 1 is present in a first type of film-coated granule comprising an amorphous solid dispersion including (i) Compound 1, (ii) copovidone and (iii) Vitamin E TPGS.

28. The method of claim 27, wherein the total amount of Compound 1 comprised in the first type of granule is 50 mg.

29. The method of claim 26, wherein Compound 2 is present in a second type of film-coated granule comprising an amorphous solid dispersion including (i) Compound 2, (ii) copovidone and (iii) Vitamin E TPGS and propylene glycol monocaprylate.

30. The method of claim 29, wherein the total amount of Compound 2 comprised in the second type of granule is 20 mg.
